(19) United States
(12) Patent Application Publication
(10) Pub. No.: US 2022/0401482 A1
(43) Pub. Date: Dec. 22, 2022

(54) GENERATION OF CD38 KNOCK-OUT PRIMARY AND EXPANDED HUMAN NK CELLS

(71) Applicants: RESEARCH INSTITUTE AT NATIONWIDE CHILDREN'S HOSPITAL, Columbus, OH (US); THE JOHNS HOPKINS UNIVERSITY, Baltimore, MD (US)

(72) Inventors: Dean Anthony LEE, Canal Winchester, OH (US); Meisam Naeimi Kararoudi, Columbus, OH (US); Gabriel Ghiaur, Baltimore, MD (US); Yuya Nagai, Baltimore, MD (US)

(21) Appl. No.: 17/773,755

(22) PCT Filed: Nov. 2, 2020
(86) PCT No.: PCT/US2020/058565

§ 371 Date: May 2, 2022

Related U.S. Application Data
(60) Provisional application No. 62/928,524, filed on Oct. 31, 2019.

(51) Int. Cl. A61K 35/17; C12N 15/90; C12N 5/0783; A61P 35/00; A61K 39/395; C07K 16/28

(52) U.S. Cl. CPC A61K 35/17; C12N 15/907; C12N 5/0646; A61P 35/00; A61K 39/39558; C07K 16/2896; C12N 2510/00; C07K 2317/24; C07K 2317/732

(57) ABSTRACT

Disclosed are genetically modified NK cells comprising a knockout of the cluster of differentiation 38 (CD38) gene and methods of using the same to treat a cancer including, but not limited to multiple myeloma, acute myeloid leukemia (AML), T-cell acute lymphoblastic leukemia (T-ALL), or Blastic plasmacytoid dendritic cell neoplasm (BPDCN).

### I. CROSS-REFERENCE TO RELATED APPLICATIONS

This application claims the benefit of U.S. Provisional Application No. 62/928,524, filed Oct. 31, 2019, which is expressly incorporated herein by reference.

### II. BACKGROUND

Cancer immunotherapy has been advanced in recent years. Genetically-modified chimeric antigen receptor (CAR) T cells are an excellent example of engineered immune cells successfully deployed in cancer immunotherapy. These cells were recently approved by the FDA for treatment against CD19⁺ B cell malignancies, but success has so far been limited to diseases bearing a few targetable antigens, and targeting such limited antigenic repertoires is prone to failure by immune escape. Furthermore, CAR T cells have been focused on the use of autologous T cells because of the risk of graft-versus-host disease caused by allogeneic T cells. In contrast, NK cells are able to kill tumor targets in an antigen-independent manner and do not cause GvHD, which makes them a good candidate for cancer immunotherapy. When combined with an antibody, the targeting and effector mechanisms of NK cells and antibody are similar to that of CAR T cells. Unfortunately, for some cancers, present treatments not only target the cancer, but can also deplete the patient's NK cell population. Daratumumab, for example, targets CD38 which is found in elevated levels on multiple myeloma cells and leukemia. The anti-tumor activity of Daratumumab is dependent on NK cells. However, CD38 is also expressed in high levels on the surface of NK cells and administration of Daratumumab results in NK cell fratricide, limiting the effectiveness of Daratumumab. Accordingly, what are needed are new immunotherapeis and/or treatment methods that can overcome the problems of NK cell fratricide.

### III. SUMMARY

Disclosed are methods and compositions related to genetically modified NK cells comprising a knockout of the CD38 gene.

In one aspect, disclosed herein are methods of treating, reducing, inhibiting, decreasing, ameliorating and/or preventing a cancer and/or metastasis (such as, for example, multiple myeloma, leukemia (including, but not limited to acute myeloid leukemia (AML), T-cell acute lymphoblastic leukemia (T-ALL), or Blastic plasmacytoid dendritic cell neoplasm (BPDCN)) in a subject comprising administering to the subject an NK cell that has been modified to comprise a knockout of the CD38 gene. In some aspects, the method of treating, reducing, inhibiting, decreasing, ameliorating and/or preventing a cancer and/or metastasis can further comprise administering to the subject a small molecule, antibody, peptide, protein, or siRNA that targets CD38 (such as for example, an anti-CD38 antibody including, but not limited to Daratumumab, isauxinmab, TAK-079, and/or MOR202).

Also disclosed herein are methods of treating, reducing, inhibiting, decreasing, ameliorating and/or preventing a cancer and/or metastasis (such as, for example, multiple myeloma, leukemia (including, but not limited to acute myeloid leukemia (AML), T-cell acute lymphoblastic leukemia (T-ALL), or Blastic plasmacytoid dendritic cell neoplasm (BPDCN)) of any preceding aspect, further comprising adrinistering to the subject an angiogenesis inhibitor (such as, for example, Pornalidomide. Lenalidornide, or Apremilast) and a steroid (such as, for example a glucocorticoid including, but not limited to dexamethasone, betamethasone prednisolone, methodiprenisolone, triarmcinolone, or fludrocortisone acetate).

In one aspect, disclosed herein are methods of treating, reducing, inhibiting, decreasing, ameliorating and/or preventing a cancer and/or metastasis that does not express CD38 directly, but in which other cells in the cancer microenvironment may be targeted (including, but not limited to myeloid-derived suppressor cells (MDSC)) comprising administering to the subject an NK cell that has been modified to comprise a knockout of the CD38 gene.

In one aspect, disclosed here are methods of genetically modifying an NK cell (such as, for example a primary or expanded NK cell) comprising obtaining guide RNA (gRNA) specific for a target DNA sequence in the NK cell (such as, for example, CD38); and b) introducing via electroporation into a target NK cell, a ribonucleoprotein (RNP) complex comprising a class 2 CRISPR/Cas endonuclease (Cas9) complexed with a corresponding CRISPR/Cas guide RNA that hybridizes to the target sequence within the genomic DNA of the NK cell creating a CD38 knockout primary or expanded NK cell.

Also disclosed herein are methods of any preceding aspect wherein the genome of the NK cell is modified by insertion or deletion of one or more base pairs, by insertion of a heterologous DNA fragment (e.g., the donor polynucleotide), by deletion of an endogenous DNA fragment, by inversion or translocation of an endogenous DNA fragment, or a combination thereof.

In one aspect, disclosed herein are methods of genetically modifying an NK cell of any preceding aspect, wherein the NK cells (for example, primary or expanded NK cells) are incubated in the presence of IL-2 and/or irradiated feeder cells for 4, 5, 6, or 7 days prior to transduction (such as, electroporation).

Also disclosed herein are methods of genetically modifying an NK cell of any preceding aspect, further comprising expanding the modified NK cells with irradiated membrane bound interleukin-21 (mbIL-21) expressing feeder cells following electroporation.

In one aspect, disclosed herein are modified NK cells made be the method of any preceding aspect. In one aspect, the modified NK cell can comprise a knockout of the gene encoding CD38. For example, In another aspect, disclosed herein is a genetically modified NK cell comprising a knockout of the gene encoding clustering of differentiation 38 (CD38) as disclosed herein for use as a medicament, preferably for use in a method of treating, reducing, inhibiting, decreasing, ameliorating and/or preventing a cancer and/or metastasis. In another aspect, disclosed herein is a genetically modified NK cell comprising a knockout of the gene encoding clustering of differentiation 38 (CD38) as disclosed herein for use in (i) a method of increasing oxidative respiration capacity in a subject in need thereof or (ii) a method of limiting extracellular NAD hydrolysis and to improve redox respiration capacity in a subject in need thereof. In the same manner, also disclosed herein is a use of a genetically modified NK cell comprising a knockout of the gene encoding clustering of differentiation 38 (CD38) as disclosed herein in the manufacture of a medicament, preferably in the manufacture of a medicament for treating, reducing, inhibiting, decreasing, ameliorating and/or preventing a cancer and/or metastasis. In another aspect, disclosed herein is a use of a genetically modified NK cell comprising a knockout of the gene encoding clustering of differentiation 38 (CD38) as disclosed herein in the manufacture of a medicament for (i) increasing oxidative respiration capacity in a subject in need thereof or (ii) limiting extracellular NAD hydrolysis and to improve redox respiration capacity in a subject in need thereof.

Also disclosed herein are methods of treating, reducing, inhibiting, decreasing, ameliorating and/or preventing a cancer and/or metastasis (such as, for example, multiple myeloma, leukemia (including, but not limited to acute myeloid leukemia (AML), T-cell acute lymphoblastic leukemia (T-ALL), or Blastic plasmacytoid dendritic cell neoplasm (BPDCN)) comprising administering to a subject with a cancer the modified NK cell of any preceding aspect (such as a CD38 knockout NK cell). In some aspects, the method of treating, reducing, inhibiting, decreasing, ameliorating and/or preventing a cancer and/or metastasis can further comprise administering to the subject a small molecule, antibody, peptide, protein, or siRNA that targets CD38 (such as for example, an anti-CD38 antibody including, but not limited to Daratumumab, isatuximab. TAK-079, and/or MOR202).

In one aspect, disclosed herein are methods of treating, reducing, inhibiting, decreasing, ameliorating and/or preventing a cancer and/or metastasis (such as, for example, multiple myeloma, leukemia (including, but not limited to acute myeloid leukemia (AML), T-cell acute lymphoblastic leukemia (T-ALL), or Blastic plasmacytoid dendritic cell neoplasm (BPDCN)) comprising administering to the subject the modified NK cell of any preceding aspect (such as, for example, a CD38 knockout NK cell) and a small molecule, antibody, peptide, protein, or siRNA that targets CD38 (such as for example, an anti-CD38 antibody including, but not limited to Daratumumab, isatuximab, TAK-079, and/or MOR202), further comprising administering to the subject an angiogenesis inhibitor (such as, for example, Pomalidomide, Lenalidomide, or Apremilast) and a steroid (such as, for example a glucocorticoid including, but not limited to dexamethasone, betamethasone, prednisolone, methodlprenisolone, triamcinolone, or fludrocortisone acetate).

Also disclosed herein are methods of reducing NK cell fratricide in a subject receiving anti-CD38 immunotherapy comprising administering to the subject the genetically modified NK cell of any preceding aspect. In one aspect, the anti-CD38 immunotherapy can comprise administering to the subject an anti-CD38 antibody including, but not limited to Daratumumab, isatuximab, TAK-079, and/or MOR202.

In one aspect, disclosed herein are methods of adoptively transferring an engineered NK cells to a subject in need thereof said method comprising a) obtaining a target NK cell (such as a primary NK cell or expanded NK cell) to be modified; b) obtaining gRNA specific for a target DNA sequence; c) introducing via electroporation into the target NK cell, a RNP complex comprising a class 2 CRISPR/Cas endonuclease (Cas9) complexed with a corresponding CRISPR/Cas gRNA that hybridizes to the target sequence within the genomic DNA of the target NK cell creating an engineered NK cell (such as, for example, an NK cell that has been modified to knockout the CD38 gene); and d) transferring the engineered NK cell into the subject.

Also disclosed herein are methods of adoptively transferring an engineered NK cells to a subject in need thereof wherein the NK cell is a primary NK cell (such as, for example, an autologous NK cell, or NK cell from an allogeneic donor source) that has been modified ex vivo and after modification transferred to the subject (such as, for example, an NK cell that has been modified to knockout the CD38 gene).

In one aspect, disclosed herein are methods of adoptively transferring an engineered NK cells to a subject in need thereof of any preceding aspect, wherein the NK cell is expanded in vitro, such as with irradiated mbIL-21 expressing feeder cells, or in vivo, such as the administration of IL-21 prior to, concurrent with, or following administration of the modified NK cells to the subject.

In one aspect, disclosed herein are methods of adoptively transferring an engineered NK cells (such as, for example, a CD38 knockout NK cell) to a subject in need thereof, wherein the subject receiving the adoptively transferred modified NK cells has a cancer.

Also disclosed herein are methods of increasing the efficacy of anti-CD38 immunotherapy (such as, for example, a small molecule, antibody (including, but not limited to Daratumumab, isatuximab, TAK-079, and/or MOR202), peptide, protein, or siRNA that targets CD38) administered to a subject for treating, reducing, inhibiting, decreasing, ameliorating and/or preventing a cancer and/or metastasis (such as, for example, multiple myeloma, leukemia (including, but not limited to acute myeloid leukemia (AML), T-cell acute lymphoblastic leukemia (T-ALL), or Blastic plasmacytoid dendritic cell neoplasm (BPDCN)) comprising administering to the subject the modified NK cell of any preceding aspect (such as, for example, a CD38 knockout NK cell).

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows CD38 expression on wild-type and CD38-knock out NK cells.

FIG. 2 shows a comparison of the resistance to daratumumab-mediated fratricide in wild-type and CD38 knockout NK cells.

FIG. 3 shows the calculated changes in ADCC (left) and overall effectiveness in killing multiple myeloma (right), based on data from FIGS. 4-6.

FIG. 4 shows the effectiveness in killing RPMI 8226 human multiple myeloma cells at various NK cell ratios for both wildtype and CD38 knockout NK cells in the presence or absence of daratumumab.

FIG. 5 shows the effectiveness in killing MM.1s human multiple myeloma cells at various NK cell ratios for both wildtype and CD38 knockout NK cells in the presence or absence of daratumumab.

FIG. 6 shows the effectiveness in killing H929 human multiple myeloma cells at various NK cell ratios for both wildtype and CD38 knockout NK cells in the presence or absence of daratumumab.

FIGS. 7A-7B show immunophenotype of ex vivo expanded NK cells. FIG. 7A shows the purity of NK cells 14 days after stimulation is shown. FIG. 7B shows representative FACS analyses of CD16 expression on CD38^{WT} and CD38^{KO} NK cells of the same donor are shown. Each figure indicates the percentage of CD16 expressing NK cells. Isotype controls are depicted with filled histogram.

FIGS. 8A-8D show successful generation of CD38^{KO} NK cells from ex vivo expanded peripheral blood NK (PB-NK) cells using Cas9 ribonucleoprotein complexes (Cas9/RNP).

FIG. 9 shows expression of genes affected by Cas9/RNP. Relative mRNA expression by RNA-seq of highly-affected genes from Table 3.

FIGS. 10A-10F show resistance of CD38^{KO} NK cells to DARA-induced fratricide. FIG. 10A shows representative FACS analyses of the conjugation assay. FIG. 10B shows summarized data of conjugation assays are shown (n=3, mean±SD). FIG. 10C shows representative FACS analyses of the fratricide assay. FIG. 10D shows viability of CD38^{WT} and CD38^{KO} NK cells treated with DARA compared to that of control samples (n=3, mean±SD). FIG. 10E shows representative FACS analyses of peripheral blood (PB) of NSG mice 7 days after treatment with DARA or saline. FIG. 10F shows summarized data of NK cell persistence in NSG mice during treatment. The frequency of human NK cells in PB at day 7 and their absolute number in spleen and bone marrow at day 9 are shown (n=5, mean±SD).

FIGS. 11A-11D show resistance of CD16^{KO} NK cells to DARA-induced fratricide. FIG. 11A shows FACS analyses of CD16^{WT} and CD16^{KO} NK cells. FIG. 11B shows that H929 cell line was incubated with CD16^{WT} or CD16^{KO} NK cells in the presence or absence of DARA (10 μg/ml) for 4 hours. FIG. 11C shows that the ADCC activity of paired CD16^{WT} and CD16^{KO} NK cells against H929 cell line in presence of DARA are shown. FIG. 11D shows that CD16^{KO}NK cells cultured in the presence of DARA (10 μg/ml) for 4 or 24 hours do show evidence fratricide.

FIGS. 12A-12F show enhanced DARA-mediated ADCC activity of CD38^{KO}NK cells against MM cell lines and primary MM cells. FIG. 12A shows representative FACS analyses of CD38 expression of NK cells and myeloma cell lines. Each figure shows the mean fluorescence intensity (MFI). Isotype controls are depicted with filled histogram. Representative data of cytotoxicity and DARA-mediated ADCC activity of paired CD38^{WT} and CD38^{KO} NK cells against myeloma cell lines (FIGS. 12B-12C) and a representative primary MM sample (FIG. 12D). FIG. 12E shows ADCC activity of paired CD38^{WT} and CD38^{KO} NK cells against primary MM samples (E:T ratio is 0.1:1). FIG. 12F shows cytotoxicity of paired CD38^{WT} and CD38^{KO} NK cells against primary DARA-resistant MM cells in the presence of DARA.

FIG. 13 shows the improvement in ADCC of CD38^{KO} NK cells compared to CD38^{WT} NK cells correlates with the level of CD38 expression on the MM cell targets. X axis indicates the relative ratio of MFI (CD38) of target cells to CD38^{WT} NK cells. Y axis indicates the relative increase in ADCC of CD38^{KO} NK cells compared to that of CD38^{WT} NK cells. The values of ADCC (E/T=5, in 4 hour-assay) are used for MM.1S, H929, and OPM-2, and those (E/T=0.1, in 24 hour-assay) are used for RPMI8226 and patient samples. Spearman's rank-correlation coefficients (r) and p value are presented.

FIG. 14 shows CD38 expression on MM cell lines 48 hours after incubation with ATRA. Control and ATRA treated samples are shown with black and gray line respectively. Unstained controls are depicted with filled histogram.

FIGS. 15A-15G show inhibitory effects of ATRA on DARA-mediated NK cell cytotoxicity. FIGS. 15A-15B show cytotoxicity and DARA-mediated ADCC activity of paired CD38^{WT} and CD38^{KO} NK cells against myeloma cell lines pretreated with 50 nM ATRA for 48 hours (mean±SD). In FIG. 15C, left panel shows representative FACS analyses data of CD38 expression on NK cells (CD3⁻CD56⁺ cells) from patients during ATRA treatment or no therapy. Frozen peripheral blood mononuclear cells were thawed and analyzed at once. Right panel shows fold increase of MFI (CD38) of NK cells during ATRA therapy compared with no therapy for 3 different patients. FIG. 15 shows representative FACS analyses data of CD38 expression on CD38^{WT} and CD38^{KO} NK cells 48 hours after incubation with 50 nM ATRA or solvent control. Control and ATRA treated samples are shown with black and gray line respectively. Unstained controls are depicted with filled histogram. FIG. 15E shows viability of CD38^{WT} and CD38^{KO} NK cells treated with DARA for 48 hours in the presence of 50 nM ATRA or solvent control compared to that of control samples (mean±SD). FIGS. 15F-15G Cytotoxicity and DARA-mediated ADCC activity of paired CD38^{WT} and CD38^{KO} NK cells against myeloma cell lines in a 48 hour-cytotoxicity assay in the presence of 50 nM ATRA or solvent control. E:T ratio is 0.25:1 for MM.1S and 0.5:1 for KMS-11 (mean±SD).

FIGS. 16A-16D show favorable metabolic reprogramming of CD38^{KO} NK cells. FIG. 16A shows that heatmap of DEGs of significantly altered pathways (cholesterol biosynthesis and OXPHOS) as determined by Ingenuity Pathway Analysis (IPA), based on normalized RNA-seq data of paired CD38^{WT} and CD38^{KO} NK cells (n=6). FIG. 16B shows principle-components analysis of DEGs, showing consistent effect of CD38 deletion for each donor despite wide inter-donor variability. FIG. 16C shows summarized data of metabolic analysis of paired CD38^{WT} and CD38^{KO} NK cells (n=3, mean±SD). FIG. 16D shows graphical analysis of basal OCR, ECAR, OCR/ECAR and SRC derived from FIG. 16C. All experiments were achieved using quintuplicate. FIGS. 16E-16F show the analysis of the additional three donors, including OCR and SRC (FIG. 16E), and ECAR and glycolytic reserve (FIG. 16F). FIG. 16G shows the analysis OCR and SRC of all six donors.

FIG. 17 shows volcano plot of normalized RNA-seq data of 6 different pairs of CD38^{WT} and CD38^{KO} NK cells. The most significantly changed genes in CD38^{KO} NK cells compared to CD38^{WT} NK cells are shown.

FIG. 18 shows CD38 expression of MM cells from a relapsed case during DARA treatment. NK cells and BMSCs from a DARA-resistant case were stained with multi-epitope anti-CD38 antibodies labeled with FITC, and then stained with anti-FITC antibody labeled with APC. MM cells were defined with CD138+ cells. MFI (CD38) of stained samples and fluorescence minus one (FMO) (without multi-epitope anti-CD38 antibodies) controls are shown.

FIG. 19 that deletion of CD38 reduces NAD recycling, resulting in increased NAD/NADH ratios.

FIG. 20 shows that, despite significant metabolic shift toward oxidative metabolism suggestive of increased mitochondrial activity, CD38 knockout does not alter mitochondrial membrane potential.

FIG. 21 shows that, despite a significant metabolic shift toward oxidative metabolism, CD38 knockout does not alter NK cell function in low-glucose settings.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

A DNA sequence that “encodes” a particular RNA is a DNA nucleic acid sequence that is transcribed into RNA. A DNA polynucleotide may encode an RNA (mRNA) that is translated into protein (and therefore the DNA and the mRNA both encode the protein), or a DNA polynucleotide may encode an RNA that is not translated into protein (e.g. tRNA, rRNA, microRNA (miRNA), a “non-coding” RNA (ncRNA), a guide RNA, etc.).

A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The term “naturally-occurring” or “unmodified” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is wild type (and naturally occurring).

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHODS OF TREATING A CANCER COMPRISING ADMINISTERING CD38 KNOCKOUT NK CELLS

Multiple Myeloma is one of the most frequently diagnosed hematologic cancers. Recently, the FDA has approved daratumumab, a therapeutic monoclonal antibody, for treating multiple myeloma. Daratumumab binds to the CD38 molecule on target cancer cells and mediates cell killing through antibody-dependent cellular cytotoxicity (ADCC) and other mechanisms. Other anti-CD38 antibodies including isatuximab, TAK-079, and MOR202 are also being developed for treatment of cancer. The ADCC induced by these antibodies is mediated by NK cells. However, the use of agents that target CD38 has a negative effect that limits its effectiveness. Because NK cells express CD38 on their cell surface, the use of these anti-CD38 antibodies result in killing of NK cells (fratricide) that are an active component to their effectiveness.

To overcome the problem of fratricide of NK cells by these anti-CD38 therapies and to treat cancer, it was recognized administration of NK cells that have been engineered to knockout expression of CD38 could address both of these issues. In short, the engineered NK cells, being resistant to the NK cell depletion caused by the anti-CD38 immunotherapy increase the efficacy anti-CD38 antibodies. Thus, in one aspect, disclosed herein are methods of increasing the efficacy of anti-CD38 immunotherapy (such as, for example, a small molecule, antibody (including, but not limited to Daratumumab, isatuximab, TAK-079, and/or MOR202), peptide, protein, or siRNA that targets CD38) administered to a subject for treating, reducing, inhibiting, decreasing, ameliorating and/or preventing a cancer and/or metastasis (such as, for example, multiple myeloma, leukemia (including, but not limited to acute myeloid leukemia (AML), T-cell acute lymphoblastic leukemia (T-ALL), or Blastic plasmacytoid dendritic cell neoplasm (BPDCN)) comprising administering to the subject the modified NK cell of any preceding aspect (such as, for example, a CD38 knockout NK cell).

It is understood and herein contemplated that this increased efficacy is profoundly beneficial to cancer patients. Accordingly, in one aspect, disclosed herein are methods of treating, reducing, inhibiting, decreasing, ameliorating and/or preventing a cancer and/or metastasis (such as, for example, multiple myeloma, leukemia (including, but not limited to acute myeloid leukemia (AML), T-cell acute lymphoblastic leukemia (T-ALL), or Blastic plasmacytoid dendritic cell neoplasm (BPDCN)) in a subject comprising administering to the subject an NK cell that has been modified to comprise a knockout of the CD38 gene. In some aspects, the method of treating, reducing, inhibiting, decreasing, ameliorating and/or preventing a cancer and/or metastasis can further comprise administering to the subject a small molecule, antibody, peptide, protein, or siRNA that targets CD38 (such as for example, an anti-CD38 antibody including, but not limited to Daratumumab, isatuximab, TAK-079, and/or MOR202).

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

It is understood and herein contemplated that where the method of treating, reducing, inhibiting, decreasing, ameliorating and/or preventing a cancer and/or metastasis (such as for example, multiple myeloma, AML, T-ALL, and/or BPDCN) comprising administering genetically modified NK cells (such as, any of the CD38 knockout NK cells disclosed herein) and an anti-CD38 agent (such as for example, Daratumumab, isatuximab, TAK-079, and/or MOR202) and/or an angiogenesis inhibitor (such as, for example, Pomalidomide, Lenalidomide, or Apremilast) and a steroid (such as, for example a glucocorticoid including, but not limited to dexamethasone, betamethasone, prednisolone, methylprednisolone, triamcinolone, or fludrocortisone acetate), and/or ATRA, the genetically modified NK cells can be administered before, current with, and/or following the administration of any anti-CD38 agent. In some aspects, the genetically modified NK cells (such as, any of the CD38 knockout NK cells disclosed herein) can be administered to the subject 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 105, 110, 115, 120 minutes, 3, 4, 5, 6, 7, 8, 9, 10, 11, 12, 13 14, 15, 16 17, 18, 19, 20, 21, 22, 23, 24, 30, 36, 42, 48, 54, 60, 66, 72, 78, 84, 90, 96 hours, 5, 6, 7, 8, 9 10, 11, 12, 13, 14, 15, 16, 17, 18, 19, 20, 21, 22, 23, 24, 25, 26, 27, 28, 29, 30, 31, 35, 42, 49, 54, or 60 days before or after administration of any anti-CD38 agent.

Also disclosed herein are methods of treating, reducing, inhibiting, decreasing, ameliorating and/or preventing a cancer and/or metastasis (such as, for example, multiple myeloma, leukemia (including, but not limited to acute myeloid leukemia (AML), T-cell acute lymphoblastic leukemia (T-ALL), or Blastic plasmacytoid dendritic cell neoplasm (BPDCN)) as set forth herein, further comprising administering to the subject an angiogenesis inhibitor (such as, for example, Pomalidomide, Lenalidomide, or Apremilast) and a steroid (such as, for example a glucocorticoid including, but not limited to dexamethasone, betamethasone, prednisolone, methodlprenisolone, triamcinolone, or fludrocortisone acetate).

It is understood and herein contemplated that the disclosed modified NK cell and adoptive transfer methods of the modified NK cells can be effective immunotherapy against a cancer. The disclosed methods and compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia (including, but not limited to AML), T-cell acute lymphoblastic leukemia (T-ALL), bladder cancer, Blastic plasmacytoid dendritic cell neoplasm (BPDCN), brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, multiple myeloma, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. Accordingly, disclosed herein, in one aspect, are methods of treating, reducing, inhibiting, decreasing, ameliorating and/or preventing a cancer and/or metastasis in a subject comprising administering to the subject an NK cell that has been modified to comprise a knockout of the CD38 gene. Accordingly, disclosed herein, in one aspect, are methods of treating, reducing, inhibiting, decreasing, ameliorating and/or preventing a cancer and/or metastasis (such as, for example multiple myeloma, leukemia (including, but not limited to acute myeloid leukemia (AML), T-cell acute lymphoblastic leukemia (T-ALL), or Blastic plasmacytoid dendritic cell neoplasm (BPDCN)) or metastasis in a subject comprising administering to the subject an NK cell that has been modified to comprise a knockout of the CD38 gene. In some aspects the methods can further comprise administering to the subject an agent that targets CD38 (such as, for example, an anti-CD38 including, but not limited to Daratumumab, isatuximab, TAK-079, and MOR202). Additionally, the methods can comprise also administering to the subject an angiogenesis inhibitor (such as for example, Pomalidomide, Lenalidomide, or Apremilast) and a glucocorticoid (such as, for example, dexamethasone, betamethasone, prednisolone, methodlprenisolone, triamcinolone, or fludrocortisone acetate).

In some embodiments of said NK cell for use or said use of an NK cell as disclosed herein, said NK cell is for administration in combination with an anti-cancer agent selected from (i) a small molecule, antibody, peptide, or protein that targets CD38 (such as for example, an anti-CD38 antibody including, but not limited to Daratumumab, Isatuximab, MOR202 and/or TAK-079) and/or (ii) an angiogenesis inhibitor (such as, for example, Pomalidomide, Lenalidomide, or Apremilast) and a steroid (such as, for example a glucocorticoid including, but not limited to dexamethasone, betamethasone, prednisolone, methylprednisolone, triamcinolone, or fludrocortisone acetate), and/or (iii) ATRA.

In embodiments, the anti-cancer agent is an anti-CD38 antibody such as isatuximab or daratumumab.

### C. METHODS OF GENETICALLY MODIFYING NK CELLS

CRISPR/CRISPR associated (Cas) protein 9 (Cas9) technology has been used recently in engineering immune cells, but genetically reprogramming NK cells with plasmids has always been challenging due to difficulties in transgene delivery in a DNA dependent manner such as lentiviral and retroviral transduction causing substantial procedure-associated NK cell apoptosis and the limited production of genetically engineered NK cells. Described herein are methods for using a DNA-free genome editing of primary and expanded human NK cells utilizing endonuclease ribonucleoprotein complexes (such as, for example, Cas9/RNPs) to reprogram (i.e., engineer or modify) NK cells.

Endonuclease/RNPs (for example, a Cas9/RNP) are comprised of three components, recombinant endonuclease protein (for example, a Cas9 endonuclease) complexed with a CRISPR loci. The endonuclease complexed to the CRISPR loci can be referred to as a CRISPR/Cas guide RNA. The CRISPR loci comprises a synthetic single-guide RNA (gRNA) comprised of a RNA that can hybridize to a target sequence complexed complementary repeat RNA (crRNA) and trans complementary repeat RNA (tracrRNA). Accordingly the CRISPR/Cas guide RNA hybridizes to a target sequence within the genomic DNA of the cell. In some cases, the class 2 CRISPR/Cas endonuclease is a type II CRISPR/Cas endonuclease. In some cases, the class 2 CRISPR/Cas endonuclease is a Cas9 polypeptide and the corresponding CRISPR/Cas guide RNA is a Cas9 guide RNA. These Cas9/RNPs are capable of cleaving genomic targets with higher efficiency as compared to foreign DNA-dependent approaches due to their delivery as functional complexes. Additionally, rapid clearance of Cas9/RNPs from the cells can reduce the off-target effects such as induction of apoptosis. Accordingly, in one aspect, disclosed here are methods of genetically modifying an NK cell comprising obtaining guide RNA (gRNA) specific for a target DNA sequence in the NK cell; and b) transducing (for example, introducing via electroporation) into a target NK cell, a ribonucleoprotein (RNP) complex comprising a class 2 CRISPR/Cas endonuclease 9 (Cas9) complexed with a corresponding CRISPR/Cas guide RNA that hybridizes to the target sequence within the genomic DNA of the NK cell.

It is understood and herein contemplated that the endonuclease used herein is note limited to the Cas9 of Streptococcus pyogenes (SpCas9) typically used for a synthetic Cas9. In one aspect, the Cas9 can come from a different bacterial source. Substitution of the Cas9 can also be used to increase the targeting specificity so less gRNA needs to be used. Thus, for example, the Cas9 can be derived from Staphylococcus aureus (SaCas9), Acidaminococcus sp. (AsCpf1), Clustered Regularly Interspaced Short Palindromic Repeats from Prevotella and Francisella 1 (Cpf1) derived from Lachnospiracase bacterium (LbCpf1), Neisseria meningitidis (NmCas9), Streptococcus thermophilus (StCas9), Campylobacter jejuni (CjCas9), enhanced SpCas9 (eSpCas9), SpCas9-HF1, Fok1-Fused dCas9, expanded Cas9 (xCas9), and/or catalytically dead Cas9 (dCas9). Additionally other Cas endonucleases can be used in place of a Cas9 system such as, for example, CasX, CasY, Cas14, Cas4, Csn2, Cas13a, Cas13b, Cas13c, Cas13d, C2c1, or C2c3 or using any other type of engineered Cas protein including prime editing.

It is understood and herein contemplated that to target the Cas9 nuclease activity to the target site and also cleave the donor plasmid to allow for recombination of the donor transgene into the host DNA, a crispr RNA (crRNA) is used. In some cases the crRNA is combined with a tracrRNA to form guide RNA (gRNA). The disclosed plasmids use AAV integration, intron 1 of the protein phosphatase 1, regulatory subunit 12C (PPP1R12C) gene on human chromosome 19, which is referred to the AAVS1, as the target site for the integration of the transgene. This locus is a “safe harbor gene” and allows stable, long-term transgene expression in many cell types. As disruption of PPP1R12C is not associated with any known disease, the AAVS1 locus is often considered a safe-harbor for transgene targeting. Because the AAVS1 site is being used as the target location, the CRSPR RNA (crRNA) must target said DNA. Herein, the guide RNA used in the disclosed plasmids comprises GGGGCCACTAGGGACAGGAT (SEQ ID NO: 2) or any 10 nucleotide sense or antisense contiguous fragment thereof. While AAVS1 is used for exemplary purposes here, it is understood and herein contemplated that other “safe harbor genes” can be used with equivalent results and can be substituted for AAVS1 if more appropriate given the particular cell type being transfected or the transgene. Examples of other safe harbor genes, include but are not limited to C—C chemokine receptor type 5 (CCR5), the ROSA26 locus, and TRAC.

It is understood and herein contemplated that there can be size limits on the donor transgene construct size delivered to the target genome. One method of increasing the allowable size of the transgene is to create additional room by exchanging the Cas9 of Streptococcus pyogenes (SpCas9) typically used for a synthetic Cas9, or Cas9 from a different bacterial source. Substitution of the Cas9 can also be used to increase the targeting specificity so less gRNA needs to be used. Thus, for example, the Cas9 can be derived from Staphylococcus aureus (SaCas9), Acidaminococcus sp. (AsCpf1), Lachnospiracase bacterium (LbCpf1), Neisseria meningitidis (NmCas9), Streptococcus thermophilus (StCas9), Campylobacter jejuni (CjCas9), enhanced SpCas9 (eSpCas9), SpCas9-HF1, Fok1-Fused dCas9, expanded Cas9 (xCas9), and/or catalytically dead Cas9 (dCas9).

It is understood and herein contemplated that the use of a particular Cas9 can change the PAM sequence which the Cas9 endonuclease (or alternative) uses to screen for targets. As used herein, suitable PAM sequences comprises NGG (SpCas9 PAM) NNGRRT (SaCas9 PAM) NNNNGATT (NmCAs9 PAM), NNNNRYAC (CjCas9 PAM), NNAGAAW (St), TTTV (LbCpf1 PAM and AsCpf1 PAM); TYCV (LbCpf1 PAM variant and AsCpf1 PAM variant); where N can be any nucleotide; V=A, C, or G; Y=C or T; W=A or T; and R=A or G.

To make the RNP complex, crRNA and tracrRNA can be mixed at a 1:1, 2:1, or 1:2 ratio of concentrations between about 50 μM and about 500 μM (for example, 50, 60, 70, 80, 90, 100, 125, 150, 175, 200, 225, 250, 275, 300, 325, 35, 375, 400, 425, 450, 475, or 500 μM), preferably between 100 μM and about 300 μM, most preferably about 200 μM at 95 C for about 5 min to form a crRNA:tracrRNA complex (i.e., the guide RNA). The crRNA:tracrRNA complex can then be mixed with between about 20 μM and about 50μM (for example 21, 22, 23, 24, 25, 26, 27, 28, 29, 30, 31, 32, 33, 34, 35, 36, 37, 38, 39, 40, 41, 42, 43, 44, 45, 46, 47 48, 49, or 50 μM) final dilution of a Cas endonuclease (such as, for example, Cas9).

Once bound to the target sequence in the target cell, the CRISPR loci can modify the genome by introducing into the target DNA insertion or deletion of one or more base pairs, by insertion of a heterologous DNA fragment (e.g., the donor polynucleotide), by deletion of an endogenous DNA fragment, by inversion or translocation of an endogenous DNA fragment, or a combination thereof. Thus, the disclosed methods can be used to generate knock-outs or knock-ins when combined with DNA for homologous recombination. It is shown herein that transduction via electroporation of Cas9/RNPs is an easy and relatively efficient method that overcomes the previous constraints of genetic modification in NK cells.

It is understood and herein contemplated that the disclosed methods can be utilized with any cell type including natural killer cells (NK cells), T cells, B cells, macrophages, fibroblasts, osteoblasts, hepatocytes, neuronal cells, epithelial cells, and/or muscle cells. Human NK cells are a subset of peripheral blood lymphocytes defined by the expression of CD56 or CD16 and the absence of T cell receptor (CD3). NK cells sense and kill target cells that lack major histocompatibility complex (MHC)-class I molecules. NK cell activating receptors include, among others, the natural cytotoxicity receptors (NKp30, NKp44 and NKp46), and lectin-like receptors NKG2D and DNAM-1. Their ligands are expressed on stressed, transformed, or infected cells but not on normal cells, making normal cells resistant to NK cell killing. In one aspect, the target cells can be primary NK cells from a donor source (such as, for example, an allogeneic donor source for an adoptive transfer therapy or an autologous donor source (i.e., the ultimate recipient of the modified NK cells), NK cell line (including, but not limited to NK RPMI8866; HFWT, K562, and EBV-LCL), or from a source of expanded NK cells derived a primary NK cell source or NK cell line.

Prior to the transduction of the NK cells, the NK cell can be incubated in a media suitable for the propagation of NK cells. It is understood and herein contemplated that the culturing conditions can comprise the addition of cytokines, antibodies, and/or feeder cells. Thus, in one aspect, disclosed herein are methods of genetically modifying an NK cell, further comprising incubating the NK cells for 1, 2, 3, 4, 5, 6, 7, 8 9, 10, 11, 12, 13, or 14 days prior to transducing the cells in media that supports the propagation of NK cells; wherein the media further comprises cytokines, antibodies, and/or feeder cells. For example, the media can comprise IL-2, IL-12, IL-15, IL-18, and/or IL-21. In one aspect, the media can also comprise anti-CD3 antibody. In one aspect, the feeder cells can be purified from feeder cells that stimulate NK cells. NK cell stimulating feeder cells for use in the claimed invention, disclosed herein can be either irradiated autologous or allogeneic peripheral blood mononuclear cells (PBMCs) or nonirradiated autologous or PBMCs; RPM18866; HFWT, K562; K562 cells transfected with membrane bound IL-15, and 41BBL, or IL-21 or any combination thereof; or EBV-LCL. In some aspects, the NK cell feeder cells provided in combination with a solution of IL-21, IL-15, and/or 41BBL. Feeder cells can be seeded in the culture of NK cells at a 1:2, 1:1, or 2:1 ratio. The It is understood and herein contemplated that the period of culturing can be between 1 and 14 days post electroporation (i.e, 1, 2, 3, 4, 5, 6, 7, 8, 9, 10, 11, 12, 13, or 14 days), preferably between 3 and 7 days, most preferably between 4 and 6 days.

It is understood and herein contemplated that the incubation conditions for primary NK cells and expanded NK cells can be different. In one aspect, the culturing of primary NK cells prior to electroporation comprises media and cytokines (such as, for example, IL-2, IL-12, IL-15, IL-18, and/or IL-21) and/or anti-CD3 antibody for less than 5 days (for example 1, 2, 3, or 4 days). For expanded NK cells the culturing can occur in the presence of NK feeder cells (at for example, a 1:1 ratio) in addition to or in lieu of cytokines (such as, for example, IL-2, IL-12, IL-15, IL-18, and/or IL-21) and/or anti-CD3 antibody. Culturing of expanded NK cells can occur for 1, 2, 3, 4, 5, 6, 7, 8, 9, or 10 days prior to transduction. Thus, in one aspect, disclosed herein are methods of genetically modifying an NK cell comprising incubating primary NK cells for 4 days in the presence of IL-2 prior to electroporation or incubating expanded NK cells in the presence of irradiated feeder cells for 4, 5, 6, or 7 days prior to electroporation.

It is understood and herein contemplated that methods of transduction to modify NK cells in the disclosed methods is limited. Due to their immune function, NK cells are resistant to viral and bacterial vectors and the induction of NK cell apoptosis by said vectors. Thus, prior to the present methods CRISPR/Cas modification of NK cells has been unsuccessful. To circumvent problems with viral vectors, the disclosed methods transform the target NK cells using electroporation. Electroporation is a technique in which an electric field is applied to cells to increase the permeability of the cell membrane. The application of the electric filed cause a charge gradient across the membrane which draws the charged molecules such as, nucleic acid, across the cell membrane. Thus, in one aspect, disclosed herein are methods of genetically modifying an NK cell comprising obtaining guide RNA (gRNA) specific for a target DNA sequence in the NK cell; and b) introducing via electroporation into a target NK cell, a ribonucleoprotein (RNP) complex comprising a class 2 CRISPR/Cas endonuclease (Cas9) complexed with a corresponding CRISPR/Cas guide RNA that hybridizes to the target sequence within the genomic DNA of the NK cell.

Following transduction (e.g., electroporation) of the NK cell, the now modified NK cell can be propagated in a media comprising feeder cells that stimulate the modified NK cells. Thus, the modified cells retain viability and proliferative potential, as they are able to be expanded post-electroporation using irradiated feeder cells. NK cell stimulating feeder cells for use in the claimed invention, disclosed herein can be either irradiated autologous or allogeneic peripheral blood mononuclear cells (PBMCs) or nonirradiated autologous or PBMCs; RPMI8866; HFWT, K562; K562 cells transfected with membrane bound IL-15, and 41BBL, or IL-21 or any combination thereof; or EBV-LCL. In some aspects, the NK cell feeder cells provided in combination with a solution of IL-21, IL-15, and/or 41BBL. Feeder cells can be seeded in the culture of NK cells at a 1:2, 1:1, or 2:1 ratio. The It is understood and herein contemplated that the period of culturing can be between 1 and 14 days post electroporation (i.e, 1, 2, 3, 4, 5, 6, 7, 8, 9, 10, 11, 12, 13, or 14 days), preferably between 3 and 7 days, most preferably between 4 and 6 days. In some aspect, the media for culturing the modified NK cells can further comprise cytokines such as, for example, IL-2, IL-12, IL-15, IL-18, and/or IL-21.

In one aspect, it is understood and herein contemplated that one goal of the disclosed methods of genetically modifying an NK cell is to produce a modified NK cell. Accordingly, disclosed herein are genetically modified NK cells made by the disclosed methods. For example, in another aspect, disclosed herein is a genetically modified NK cell comprising a knockout of the gene encoding clustering of differentiation 38 (CD38) as disclosed herein for use as a medicament, preferably for use in a method of treating, reducing, inhibiting, decreasing, ameliorating and/or preventing a cancer and/or metastasis. In another aspect, disclosed herein is a genetically modified NK cell comprising a knockout of the gene encoding clustering of differentiation 38 (CD38) as disclosed herein for use in (i) a method of increasing oxidative respiration capacity in a subject in need thereof or (ii) a method of limiting extracellular NAD hydrolysis and to improve redox respiration capacity in a subject in need thereof. In the same manner, also disclosed herein is a use of a genetically modified NK cell comprising a knockout of the gene encoding clustering of differentiation 38 (CD38) as disclosed herein in the manufacture of a medicament, preferably in the manufacture of a medicament for treating, reducing, inhibiting, decreasing, ameliorating and/or preventing a cancer and/or metastasis cancer. In another aspect, disclosed herein is a use of a genetically modified NK cell comprising a knockout of the gene encoding clustering of differentiation 38 (CD38) as disclosed herein in the manufacture of a medicament for (i) increasing oxidative respiration capacity in a subject in need thereof or (ii) limiting extracellular NAD hydrolysis and to improve redox respiration capacity in a subject in need thereof.

As noted above, fratricide of NK cells in subjects receiving an anti-CD38 therapy such as daratumumab to treat a cancer like multiple myeloma, leukemia (including, but not limited to acute myeloid leukemia (AML), T-cell acute lymphoblastic leukemia (T-ALL), or Blastic plasmacytoid dendritic cell neoplasm (BPDCN) is a significant problem as NK cells express high levels of CD38 on their cell surface. Thus, it is understood and herein contemplated that one modification of NK cells that would be advantageous for the treatment of cancer is the knockout of CD38 to produce an NK cell population not susceptible to NK cell fratricide during anti-CD38 treatment. Such modified cells can be very useful in immunotherapy of any disease or condition that could be treated with the addition of NK cells. Thus, in one aspect, disclosed herein are genetically modified NK cell comprising a knockout of the gene encoding CD38.

As noted throughout the present disclosure, the disclosed modified NK cells are ideally suited for use in immunotherapy such as the adoptive transfer of modified (i.e, engineered NK cells to a subject in need thereof. Thus, in one aspect, disclosed herein are methods of adoptively transferring an engineered NK cells to a subject in need thereof said method comprising a) obtaining a target NK cell to be modified; b) obtaining gRNA specific for a target DNA sequence; c) introducing via electroporation into the target NK cell, a RNP complex comprising a class 2 CRISPR/Cas endonuclease (Cas9) complexed with a corresponding CRISPR/Cas gRNA that hybridizes to the target sequence within the genomic DNA of the target NK cell creating an engineered NK cell; and d) transferring the engineered NK cell into the subject.

In one aspect, the modified NK cells used in the disclosed immunotherapy methods can be primary NK cells from a donor source (such as, for example, an allogeneic donor source for an adoptive transfer therapy or an autologous donor source (i.e., the ultimate recipient of the modified NK cells), NK cell line (including, but not limited to NK RPM18866; HFWT, K562, and EBV-LCL), or from a source of expanded NK cells derived a primary NK cell source or NK cell line. Because primary NK cells can be used, it is understood and herein contemplated that the disclosed modifications of the NK cell can occur ex vivo or in vitro.

Following transduction of the NK cells, the modified NK cells can be expanded and stimulated prior to administration of the modified (i.e., engineered) NK cells to the subject. For example, disclosed herein are methods of adoptively transferring NK cells to a subject in need thereof wherein the NK cell is expanded with irradiated mbIL-21 expressing feeder cells prior to administration to the subject. In some aspect, it is understood and herein contemplated that the stimulation and expansion of the modified (i.e, engineered) NK cells can occur in vivo following or concurrent with the administration of the modified NK cells to the subject. Accordingly disclosed herein are immunotherapy methods wherein the NK cells are expanded in the subject following transfer of the NK cells to the subject via the administration of IL-21 or irradiated mbIL-21 expressing feeder cells.

1. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

2. Nucleic Acids

There are a variety of molecules disclosed herein that are nucleic acid based, including for example the nucleic acids that encode, for example CD38, or any of the nucleic acids disclosed herein for making CD38 knockouts, or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to the protein molecules involved in the signaling pathways disclosed herein, for example CD38, all of which are encoded by nucleic acids or are nucleic acids. The sequences for the human analogs of these genes, as well as other analogs, and alleles of these genes, and splice variants and other types of variants, are available in a variety of protein and gene databases, including Genbank. Those of skill in the art understand how to resolve sequence discrepancies and differences and to adjust the compositions and methods relating to a particular sequence to other related sequences. Primers and/or probes can be designed for any given sequence given the information disclosed herein and known in the art.

c) Primers and Probes

Disclosed are compositions including primers and probes, which are capable of interacting with the disclosed nucleic acids, such as CD38 as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the CD38 gene typically can be used to produce an amplified DNA product that contains a region of the CD38 gene or the complete gene. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

3. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes ß-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

4. Peptides

a) Protein Variants

Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative
Substitutions, others are known in the art.

| | |
|---|---|
| Ala | Ser |
| Arg | Lys; Gin |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J Chem. Soc Perkin Trans. 1307-314 (1982) (—CH—CH—, cis and trans); Almquist et al. J Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

5. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

It is understood and herein contemplated that the disclosed modified NK cell and adoptive transfer methods of the modified NK cells can be effective immunotherapy against a cancer. The disclosed methods and compositions can be used to treat, inhibit, reduce, and/or prevent any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general. A representative but non-limiting list of cancers that the disclosed compositions can be used to treat is the following: lymphoma, B cell lymphoma, T cell lymphoma, mycosis fungoides, Hodgkin's Disease, myeloid leukemia (including, but not limited AML), T-cell acute lymphoblastic leukemia (T-ALL), bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, BPDCN, multiple myeloma, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. Accordingly, disclosed herein, in one aspect, are methods of treating, reducing, inhibiting, decreasing, ameliorating and/or preventing a cancer and/or metastasis (such as, for example multiple myeloma, leukemia (including, but not limited to acute myeloid leukemia (AML), T-cell acute lymphoblastic leukemia (T-ALL), or Blastic plasmacytoid dendritic cell neoplasm (BPDCN)) or metastasis in a subject comprising administering to the subject an NK cell that has been modified to comprise a knockout of the CD38 gene. In some aspects the methods can further comprise administering to the subject an agent that targets CD38 (such as, for example, an anti-CD38 including, but not limited to Daratumumab, isatuximab, TAK-09, and MOR202). Additionally, the methods can comprise also administering to the subject an angiogenesis inhibitor (such as for example, Pomalidomide, Lenalidomide, or Apremilast) and a glucocorticoid (such as, for example, dexamethasone, betamethasone, prednisolone, methodlprenisolone, triamcinolone, or fludrocortisone acetate) and/or ATRA.

As noted herein, the disclosed modified NK cells (such as, for example, CD38 knockout NK cells disclosed herein including but not limited to CD3S knockout NK cells made by the methods disclosed herein) can be used in treatments where anti-CD3S therapy being administered to a subject can or has resulted in NK cell fratricide. Accordingly, in one aspect, also disclosed herein are methods of reducing NK cell fratricide in a subject receiving anti-CD38 immunotherapy comprising administering to the subject any genetically modified NK cell disclosed herein (including the CD38 knockout NK cells disclosed herein). In one aspect, the anti-CD38 immunotherapy can comprise administering to the subject an anti-CD38 antibody including, but not limited to Daratumumab, isatuximab, TAK-079, and/or MOR202,

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Generating CD38-KO NK Cells to Overcome Fratricide and Enhance ADCC

Natural killer cells play an important role in targeting CD38-expressing Multiple myeloma (MM) by anti-CD38 monoclonal antibody, daratumamb (DARA). To overcome fratricide of NK cells in DARA therapy, knock-out NK cells using Cas9/RNP were generated. Specifically, gRNA sequences to target CD38 for deletion using Cas9/RNP. The gRNA targeting the following sequence (at exon 1 near the start codon of CD38) was produced commercially from Integrated DNA Technologies: 5′-CTGAACTCGCAGTTGGCCAT-3′ (SEQ ID NO: 1)

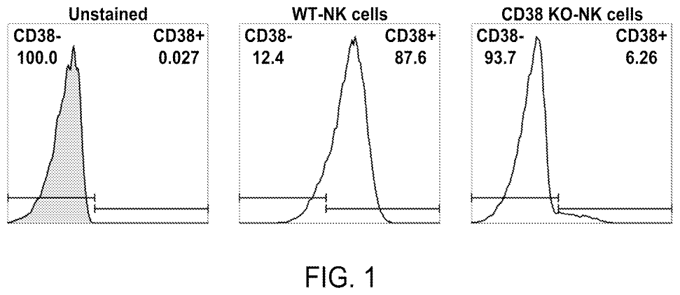

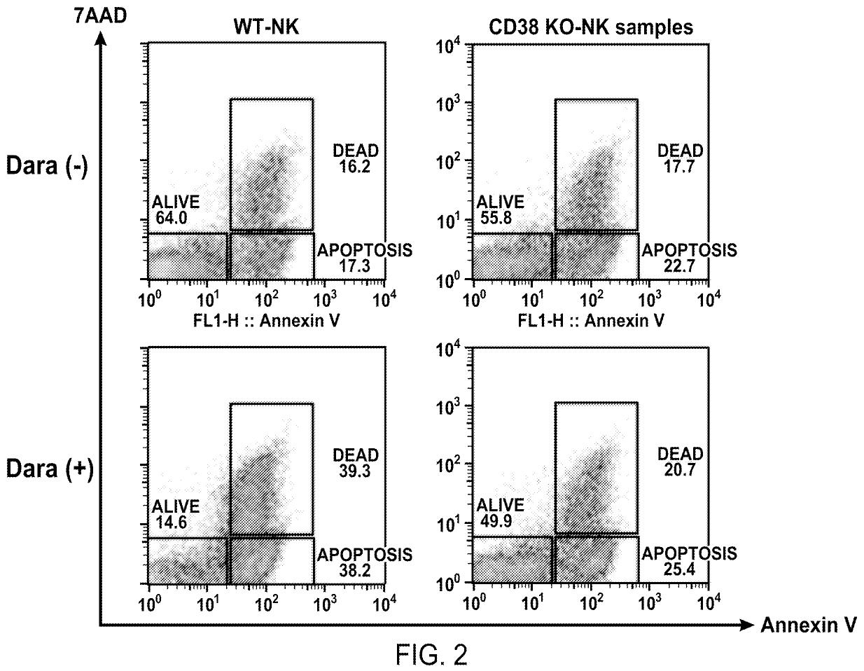

NK cells were electroporated with Cas9/RNP and expanded for 14 days. The resulting gene deletion was over 90% effective, reducing the CD38 expression in NK cells from 87% in control NK cells to 7% in CD38-deleted NK cells, without using any method for positive selection of the KO population (FIG. 1). NK cells were tested for sensitivity to daratumumab-mediated fratricide. Unmodified control NK cells showed 77% sensitivity to fratricide (viability reduced from 64% to 14.6%). In contrast, CD38-KO NK cells showed only 11% sensitivity to fratricide (viability reduced from 55.8% to 49.90%) (FIG. 2).

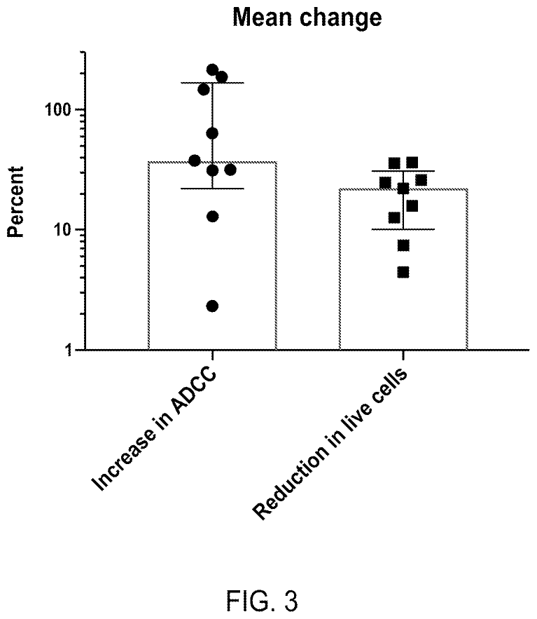

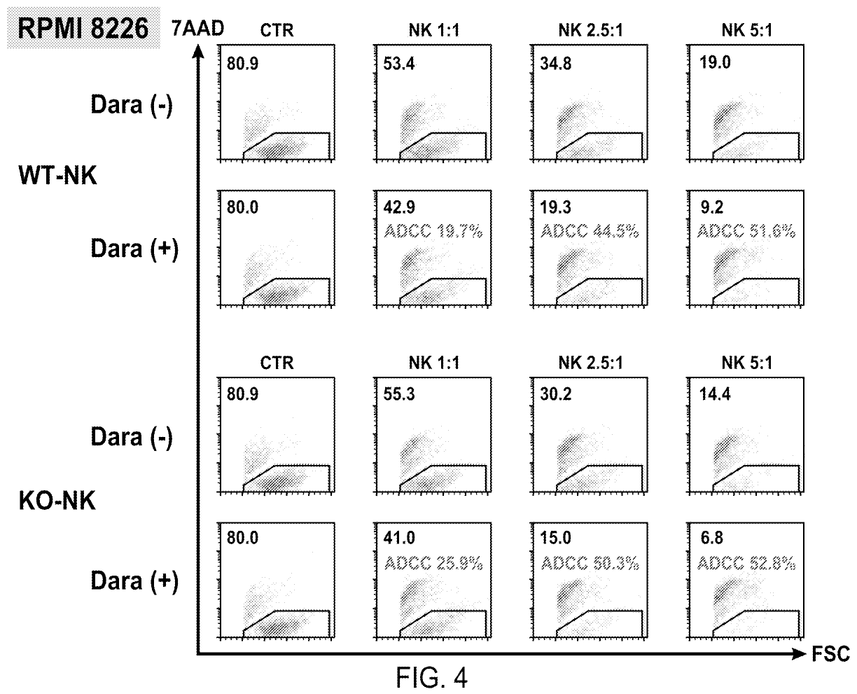

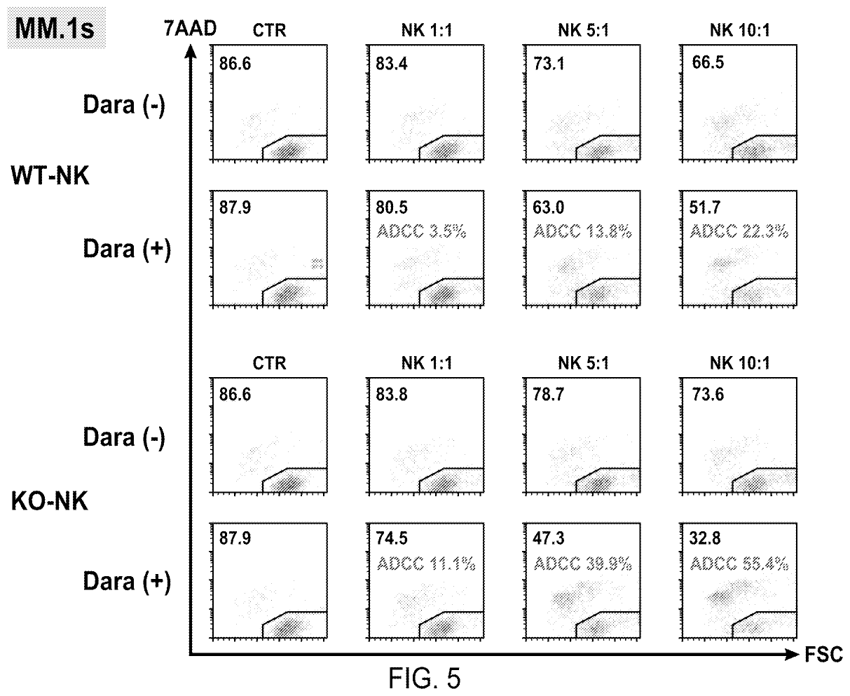

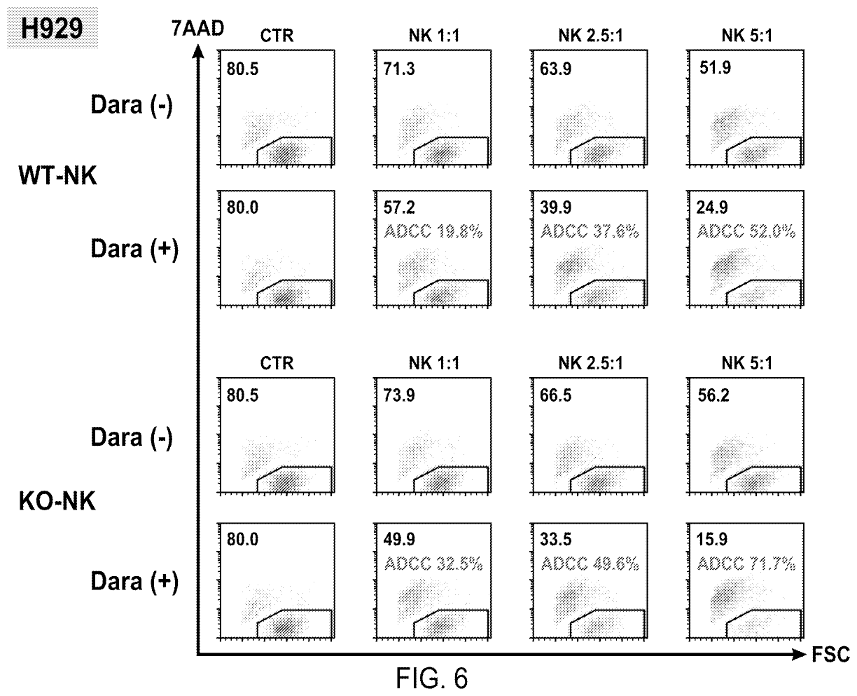

Next, NK cells were tested for improved antibody-dependent cell cytotoxicity (ADCC) against multiple myeloma. When measured across 3 different target cells and 3 E:T ratios, CD38-KO NK cells increased their ADCC by 37.9% compared to wild-type controls (median, p=0.004) and decreased final surviving cancer cell population by 22.3% (median, p=0.004) (FIG. 3). Individual results are presented in FIGS. 4-6.

### 2. Example 2: Introduction

Multiple myeloma (MM) is characterized by clonal accumulation of malignant plasma cells in bone marrow (BM). Although the introduction of autologous stem cell transplantation and novel agents such as proteasome inhibitors (PI-bortezomib/carfilzomib) as well as immunomodulatory IMiD drugs (lenalidomide/pomalidomide) has significantly improved survival in MM patients, virtually all patients relapse and then suffer from poor prognosis with median overall survival of only 13 months.

Most recently, monoclonal antibodies targeting CD38, daratumumab (DARA) and isatuximab have made a significant impact on the management of patients with MM. DARA is approved by the U.S. Food and Drug Administration (FDA) for newly diagnosed as well as relapsed/refractory patients with M. DARA kills target cells through several mechanisms: complement dependent cytotoxicity (CDC), antibody dependent cellular cytotoxicity (ADCC), antibody dependent cellular phagocytosis (ADCP), apoptosis induced by cross-linking of CD38 on the target cells, as well as immunomodulatory effects via elimination of CD38⁺ immunosuppressive cells. Although all of these actions are involved in the anti-tumor activity, it remains unclear which mechanism plays a major role in the clinical responses seen in patients with MM.

Despite the well-established clinical benefits of DARA, the majorities of patients eventually experience disease relapse and continue to succumb to MM. Current research and clinical efforts are underway to unveil mechanisms of resistance to DARA and develop combination therapies to deepen/boost the response. Clinical evidence indicates that IMiDs synergize with DARA and result in better disease control. This can be in part due to activation of natural killer (NK) cells that mediate DARA-mediated ADCC as well as IMiDs-induced CD38 up-regulation on MM cells via cereblon-mediated degradation of Ikaros/Aiolos.

Additional evidence indicates that CD38 expression levels on target cells correlates with sensitivity to DARA. MM cells with higher CD38 expression levels are preferentially killed by DARA, while residual MM cells display lower CD38 expression levels during treatment with DARA. Because transcription of CD38 is directly controlled by retinoic acid (RA) via RA responsive elements present in intron I of the CD38 gene, all-trans retinoic acid (ATRA) up-regulates CD38 expression on a variety of hematopoietic cells including MM cells. In addition, ATRA down-regulates expression of complement inhibitory proteins (CD55 and CD59) and synergizes with DARA to kill target MM cells. This strategy is currently being tested in a clinical trial combining ATRA with DARA for patients with MM (NCT02751255).

Another putative mechanism of suboptimal response to DARA is rapid depletion of NK cells in patients following treatment with DARA, because NK cells also express relatively high levels of CD38. This decrease in circulating NK cells persists for 3-6 months after discontinuation of treatment, resulting in inefficient ADCC against MM cells. Adoptive transfer of NK cells can be a strategy to overcome this mechanism. In a preclinical model, supplementation of ex vivo expanded NK cells results in a significant albeit modest improvement of DARA in controlling disease burden, because these NK cells are also subject to DARA-mediated elimination. An approach to overcome DARA-mediated elimination is to delete CD38 in NK (CD38^{KO} NK) cells. Although gene editing of NK cells has been challenging due to their DNA-sensing mechanisms and associated apoptosis, efficient gene deletion in primary NK cells can be achieved using a DNA-free method with Cas9 ribonucleoproteins complexes (Cas9/RNP).

Here, the biological consequences of CD38 deletion in NK cells were explored with regard to DARA immunotherapy by assessing conjugation and fratricide in vitro and in vivo, and ADCC against MM cells. Lastly, the role of CD38 was explored as an ectoenzyme that regulates nicotinamide adenine dinucleotide (NAD⁺) levels on NK cell metabolism and transcription.

### 3. Example 3: Materials and Methods

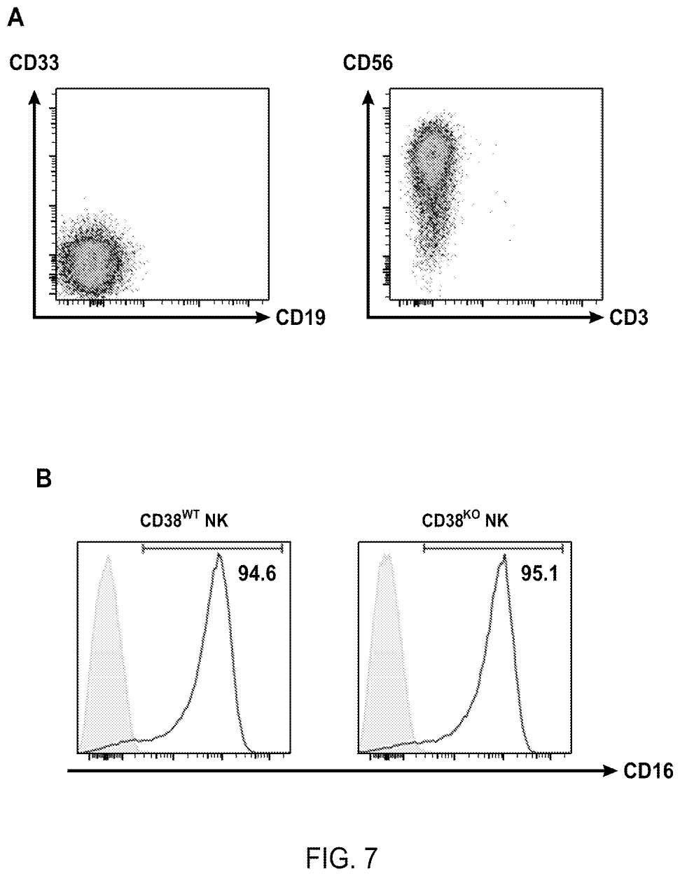

NK cells purification and expansion. Peripheral blood NK cells were isolated from healthy donors. Purified NK cells (CD3⁻/CD56⁺) were stimulated using irradiated membrane bound IL-21 (mbIL21)/4-1BBL expressing K562 (CSTX002) at a ratio of 2:1 and grown for 7 days in AIM-V/ICSR expansion medium (CTS™AIMV™ SFM/CTS™ Immune Cell SR, Thermo Fisher Scientific) and 50 IU of human recombinant IL-2 (rIL-2) (Novartis). Neither CD3⁺, CD19⁺, nor CD33⁺ cells were detected after stimulation (FIG. 7). Prior to electroporation on day six of expansion, half of the medium was changed.

Multiple myeloma cells. MM cell lines H929, MM.1S and U266 were purchased from the American Type Culture Collection. OPM-2 and KMS-11 cell lines were obtained from German Collection of Microorganisms and Cell Cultures. Primary MM cells were collected from newly diagnosed or relapsed MM patients under an IRB-approved protocol and written consent at Johns Hopkins University. Patient information is provided in Table 1. CD138⁺ MM cells purification and cell culture were previously published. Mononuclear cells were isolated from fresh BM aspirates by density gradient centrifugation using Ficoll-Paque™ PLUS (GE Healthcare Bio-Sciences AB, Sweden). CD138⁺ MM cells were selected by magnetic beads and columns according to the manufacturer's instructions (Miltenyi Biotec, Auburn, Calif.). All MM cell lines and primary MM cells were cultured in RPMI 1640 medium (Gibco, UK) supplemented with 10% heat-inactivated FBS (Corning, Manassas, Va.), 2 mM L-Glutamine (Gibco, Grand Island, N.Y.), 100 units/ml penicillin and 100 μg/ml streptomycin (Gibco, Grand Island, N.Y.).

TABLE 1

| | | | Frequency | | | | |
|---|---|---|---|---|---|---|---|
| | | | of plasma | | | | Last |
| | | | cells | | | | DARA |
| | Age | Subtype | (biopsy) | ISS | Chromosomes | Treatment history | infusion |
| Patient-1 | 75 | IgG lambda | 50% | NA | nuc ish(D3Z1, CCND1- | naïve | none |
| | | myeloma | | | XT)x3, (D9Z1, D15Z4)x3-4 | | |
| Patient-2 | 67 | IgG lambda | 70-80% | 1 | t(11;14) and 11q− | PI, IMiD, autologous | 22 |
| | | myeloma | | | | SCT, DARA | months |
| | | | | | | XPO1 inhibitor, BCL2 | before |
| | | | | | | inhibitor, anti-BCMA | |
| | | | | | | Ab | |
| Patient-3 | 47 | IgG kappa | <5% | 1 | unkown | naïve | none |
| | | plasmacytoma | | | | | |
| Patient-4 | 73 | IgA lambda | 10-15% | 1 | 1q+, 1p−, 6+, 9+, 15+, IgH | naïve | none |
| | | myeloma | | | translocation without usual | | |
| | | | | | partner | | |
| Patient-5 | 57 | IgA kappa | 40-50% | 3 | 1q+, t(4;14), −13 myc | naïve | none |
| | | myeloma | | | disruption | | |
| DARA- | 42 | IgG lambda | <1% | 3 | +1q, monosomy 13, del 14 | PI, autologous SCT, | 10 days |
| resistant | | myeloma | | | | IMiD, DARA | before |
| patient | | | | | | | |

Mice. NOD.Cg-Prkdc^{scid}Il2^{tm1WSj1}/SzJ (NSG) mice were purchased from The Jackson Laboratory, and maintained under specific pathogen-free conditions. Six- to ten-week-old male NSG mice were used for experiments in accordance with our animal protocol approved by the animal research committee at Johns Hopkins University.

Immunophenoyping. NK cells and MM cells were stained with fluorophore-conjugated antibodies. The list of antibodies is shown in Table 2. The cells were washed and analyzed by flow cytometry using LSR II flow cytometer (Becton Dickinson Biosciences) and FBowJo software (Tree Star Inc, Ashland Oreg.).

TABLE 2

| Brand | Reactivity | Name | Clone | Format |
|---|---|---|---|---|
| BD | Human | CD3 | SK7 | FITC |
| BD Pharmingen | Human | CD16 | 3G8 | PE |
| BD Pharmingen | Human | CD16 | 3G8 | Alexa Fluor 647 |
| BD Pharmingen | Human | CD19 | SJ25C1 | APC-Cy7 |
| BD Horizon | Human | CD33 | WM53 | BV421 |
| BD | Human | CD38 | HB7 | PE |
| Biolegend | Human | CD38 | HB7 | APC |
| Biolegend | Human | CD38 | HIT2 | Biotin |
| BD Pharmingen | Human | CD56 | B159 | Alexa Fluor 647 |
| BD Pharmingen | Human | CD138 | MI15 | PE |
| BD Pharmingen | Human | CD45 | HI30 | APC |
| BD Horizon | Human | CD45 | HI30 | BV421 |
| BD Pharmingen | Mouse | CD45 | 30-F11 | FITC |

Generation of CRISPR modified cells. To generate CD38^{KO} and CD16^{KO} NK cells, we used crisprRNA (crRNA) (5-CTGAACTCGCAGTTGGCCAT; SEQ ID NO: 1) targeting the exon 1 of the CD38 gene³⁴ and crRNA (5-AAAGAGACTTGGTACCCAGG; SEQ ID NO: 3) targeting the exon 5 of CD16A gene. Generating Cas9/RNP complex has been described previously. To generate CD38^{KO} and CD16^{KO} NK cells, we used crisprRNA (crRNA) (5-CTGAACTCGCAGTTGGCCAT; SEQ ID NO: 1) targeting the exon 1 of the CD38 gene and crRNA (5-AAAGAGACTTGGTACCCAGG; SEQ ID NO: 4) targeting the exon 5 of CD16A gene. Generating Cas9/RNP complex has been described previously. In brief, pre-transcribed Alt-R® CRISPR-Cas9 crRNAs and Alt-R® CRISPR-Cas9 tracrRNA (Catalog #1072532) were purchased from IDT (Integrated DNA Technologies, Inc., Coralville, Iowa). Guide RNA (gRNA) was prepared by incubating 200 μM each of crRNA and tracrRNA together in a total volume of 10 μl in Nuclease-Free IDTE, pH 7.5 (1×TE solution, Catalog #11-01-02-02) at 95° C. for 5 minutes. The Cas9/RNP complex was formed by incubating 2 μl of Alt-R® S.p. HiFi Cas9 Nuclease V3 protein (122 pmol) (Catalog #1081060), 2 μl of gRNA (400 pmol), and 1 μl of PBS in a total volume of 5 μl for 15-20 minutes at room temperature. Day 7-expanded NK cells were resuspended in 20 μl of P3 Primary Cell 4D-Nucleofector™ X Solution and 5 μl of Cas9/RNP complex and 1 μl of 100 μM of Alt-R® Cas9 Electroporation Enhancer (Catalog #1075915), and electroporated using Lonza 4D-Nucleofector system with pulse EN-138. Wild type NK (CD38WT NK) cells were electroporated without Cas9/RNP complex. After electroporation, the NK cells were rested for 2 days in AIM-V/ICSR growth medium supplemented with 50 IU of rIL-2 before assessing the efficiency of CRISPR modification using flow cytometry. The cells were then expanded with CSTX002. Residual CD38+ NK cells were removed by labeling with biotinylated anti-CD38 antibody (BioLegend) followed by anti-biotin microbeads (Miltenyi Biotec, Auburn, Calif.) and depletion on an LD column (Miltenyi Biotec, Auburn, Calif.).

Identifying off-target effects of CD38-targeted Cas9/RNP. Whole genome sequencing was used to identify the off-target effects of Cas9/RNP targeted to CD38. Genomic DNA (gDNA) was isolated form CD38^{WT} and CD38^{KO} NK cells using DNeasy Blood and Tissue Kit (Qiagen, Cat No./ID: 69504). DNA libraries were constructed using NEBNext Ultra II-FS DNA Library Prep Kit (New England Biolabs, Ipswhich Mass.). Samples were enzymatically fragmented, 5′ phosphorylated, dA-Tailed, and ligated with a unique, dual indexed adapter approach to prevent sample mis-assignment and resolve index hopping (Integrated DNA Technologies, Iowa). The adaptor-ligated DNA was amplified by limit-cycle PCR and purified using a magnetic-bead based approach. Library quality was analyzed on Tapestation High-Sensitivity D1000 ScreenTape (Agilent Biotechonologies) and quantified by KAPA qPCR (KAPA BioSystems). Libraries were sequenced at 2×150 bp read lengths to a depth of ˜30× coverage on the Illumina HiSeq4000 platform.

Identifying off-target effects of CD38-targeted Cas9/RNP. Whole genome sequencing was used to identify the off-target effects of Cas9/RNP targeted to CD38. Genomic DNA (gDNA) was isolated form CD38^{WT} and CD38^{KO} NK cells using DNeasy Blood and Tissue Kit (Qiagen, Cat No./ID: 69504). DNA libraries were constructed using NEBNext Ultra II-FS DNA Library Prep Kit (New England Biolabs, Ipswhich Mass.). Samples were enzymatically fragmented, 5′ phosphorylated, dA-Tailed, and ligated with a unique, dual indexed adapter approach to prevent sample mis-assignment and resolve index hopping (Integrated DNA Technologies, Iowa). The adaptor-ligated DNA was amplified by limit-cycle PCR and purified using a magnetic-bead based approach. Library quality was analyzed on Tapestation High-Sensitivity D1000 ScreenTape (Agilent Biotechonologies) and quantified by KAPA qPCR (KAPA BioSystems). Libraries were sequenced at 2×150 bp read lengths to a depth of ˜30× coverage on the Illumina HiSeq4000 platform.

NK function assays. NK cell conjugation assay was performed as previously published and as detailed above. To quantify fratricide, CD38^{WT} and CD38^{KO} NK cells were each treated with 10 μg/ml of DARA or solvent control for 4 or 24 hours, then stained with PO-PRO™-1 dye (Invitrogen, Eugene, Oreg.) and 7-aminoactinomycin D (7-AAD) (Invitrogen, Eugene, Oreg.)¹⁵. Flow-based killing assays were performed as detailed in Supplemental Methods. In brief, CD38^{WT} or CD38^{KO} NK cells were co-cultured with CFSE-labeled target MM cells for 4 or 24 hours in the presence of 10 μg/ml of DARA or solvent as control.

Adoptive transfer of human NK cells into NSG mice. Ex vivo expanded CD38^{WT} and CD38^{KO} NK cells from the same individual donor were thawed and re-stimulated with irradiated CSTX002 for one week. 10⁷ NK cells from each group were suspended in Hank's balanced salt solution (Gibco, Grand Island, N.Y.) and infused through tail vein into NSG mice that were pre-treated intraperitoneally with DARA (8 mg/kg) or solvent control on the same day. Mice were supplied with rIL-2 (50,000 IU) intraperitoneally every other day. Peripheral blood (after 7 days), and spleen and BM (after 9 days) were collected and analyzed for the persistence of NK cells in each mouse. Absolute numbers of human NK cells in spleen and BM from 2 femurs were also calculated.

RNA sequencing and Ingenuity Pathway Analysis. Strand-specific RNA-seq libraries were prepared using NEBNext Ultra II Directional RNA Library Prep Kit, following the manufacturer's recommendations (New England Biolabs, Ipswhich Mass.). In summary, total RNA isolated from same donors (n=6) CD38^{WT} and CD38^{KO} NK cells, (total of 12 samples) quality was assessed using RNA 6000 Nano kit on Agilent 2100 Bioanalyzer (Agilent Biotechnologies) and concentration measured using Qubit RNA HS assay kit (Life Technologies). A 40-500 ng aliquot of total RNA was rRNA depleted using NEB's Human/Mouse/Rat RNAse-H based Depletion kit (New England BioLabs). Following rRNA removal, mRNA was fragmented and then used for first- and second-strand cDNA synthesis with random hexamer primers. Double stranded cDNA fragments underwent end-repair and a-tailing and ligation of dual-unique adapters (Integrated DNA Technologies). Adaptor-ligated cDNA was amplified by limit-cycle PCR and purified using a magnetic-bead based approach. Library quality was analyzed on Tapestation High-Sensitivity D1000 ScreenTape (Agilent Biotechonologies) and quantified by KAPA qPCR (KAPA BioSystems). Libraries were pooled and sequenced at 2×150 bp read lengths on the Illumina HiSeq 4000 platform to generate approximately 60-80 million paired-end reads per sample. We next used the normalized RNA-seq data and filtered before input to Ingenuity Pathway Analysis (IPA) by eliminating genes that were not expressed at greater than or equal to 10 FPM in at least one sample. Differentially-expressed genes (DEGs) were identified as those in which a paired two-sided t-test of gene expression levels between CD38^{WT} and CD38^{KO} NK cells yielded a p-value of less than 0.05. Adjusting the p-value cutoff for DEGs to 0.01 or 0.1, or adjusting the minimum gene expression cutoff to 5 FPM, does not qualitatively affect conclusions. The mean fold changes of each gene across CD38^{WT} and CD38^{KO} NK cells are approximately equal to the fold changes of the means in the reported pathways, so inter-individual effects (CD38^{WT} and CD38^{KO} NK cells from the same donors) can be considered negligible for these conclusions. All default settings for a core analysis in IPA were implemented. We were not able to study the transcriptomic profile of CD38^{WT} and CD38^{KO} NK cells in presence of DARA, as the CD38^{WT} NK cells are killed by DARA-induced fratricide.

Differentially-expressed genes (DEGs) were identified as those in which a paired two-sided t-test of gene expression levels between CD38^{WT} and CD38^{KO} NK cells yielded a p-value of less than 0.05. Adjusting the p-value cutoff for DEGs to 0.01 or 0.1, or adjusting the minimum gene expression cutoff to 5 FPM, does not qualitatively affect conclusions. The mean fold changes of each gene across CD38^{WT} and CD38^{KO} NK cells are approximately equal to the fold changes of the means in the reported pathways, so inter-individual effects (CD38^{WT} and CD38^{KO} NK cells from the same donors) can be considered negligible for these conclusions. All default settings for a core analysis in IPA were implemented. We were not able to study the transcriptomic profile of CD38^{WT} and CD38^{KO} NK cells in presence of DARA, as the CD38^{WT}NK cells are killed by DARA-induced fratricide.

Metabolic assays. To measure oxygen consumption rate (OCR) and extracellular acidification rate (ECAR), we used the Agilent Extracellular Flux assay Kit (Agilent Technologies) on the Seahorse XFe24 (Agilent Technologies). Expanded CD38^{WT} and CD38^{KO}NK cells were incubated in XF RPMI for 1-2 hours prior to the measurements at 37° C. in a non-CO₂ incubator. The medium was supplemented with 10 μM glucose and 2 μM L-glutamine with no phenol red at a pH of 7.35-7.4. OCR, a measure of oxidative phosphorylation (OXPHOS), and ECAR were analyzed under the basal condition following by addition of 1 μM oligomycin, 1.5 μM FCCP, and 0.5 μM rotenone and antimycin A to the cell culture.

Conjugation assay, fratricide assay and flow-based cytotoxicity assay. NK cell conjugation assay was performed with minor modifications as described by Burshtyn et al. In brief, 2×10⁶ cells (1×10⁷ cell/ml) of each NK cells were stained with 5 μM of green dye CFDA SE (CFSE) cell tracer (Invitrogen, Eugene, Oreg.) for 15 minutes at 37° C. or 5 μM red dye PKH26 (Sigma-Aldrich, St. Louis, Mo.) for 5 minutes at room temperature. Staining was stopped by adding complete medium to the cell suspension. The cells were washed twice with complete medium. The green and red labeled NK cells were mixed at the ratio of 1:1 in 200 μl of total volume supplemented with 10 μg/ml of DARA or solvent control (saline) and co-cultured at 37° C. in a 5% CO₂ incubator for 4 hours. Then the cells were gently collected and fixed with 200 μl of 4% formaldehyde and 20,000 cells were analyzed for conjugation using flow cytometry.

To quantify fratricide, CD38^{WT} and CD38^{KO} NK cells were each treated with 10 μg/ml of DARA or solvent control for 4 or 24 hours, then stained with PO-PRO™-1 dye (Invitrogen, Eugene, Oreg.) and 7-aminoactinomycin D (7-AAD) (Invitrogen, Eugene, Oreg.). Viable NK cells (PO-PRO™-1 negative/7-AAD negative) were assessed based on the frequency or absolute number using beads (Becton Dickinson Biosciences).

To assess ADCC, MM cell lines or purified primary CD138⁺ MM cells were labeled with 5 μM of CFSE and co-cultured with CD38^{WT} or CD38^{KO} NK cells at the indicated effector-to-target (E:T) ratios in flat bottom 96-well plates (Falcon, USA) in the presence of 10 μg/ml of DARA or solvent as control³⁸. Due to low frequency of DARA-resistant primary samples (CD38^{negative/low}), we did not purify these cells and MM cells were defined as CD138⁺CD4⁻ cells. The viability of target cells was analyzed after 4 hours for MM cell lines, and 24 hours for primary samples. In some experiments, myeloma cell lines were pretreated with 50 nM of ATRA for 2 days prior to 4-hour cytotoxicity assay. To study the effect of ATRA on overall DARA-mediated NK cell cytotoxicity, MM cells and NK cells were co-cultured for 48 hours in the presence of DARA and 50 nM of ATRA. Viable target cells were assessed from the percent or absolute number of 7-AAD negative/CFSE positive cells among total CFSE positive cells or using beads. Background was determined from the target cells incubated in the absence of effector cells and DARA. The percentage of DARA-mediated ADCC (%) was calculated according to the formula: (1−the percent or absolute number of viable target cells in the presence of effector cells with DARA/that of the corresponding sample with solvent control)×100. All assays were performed in triplicate with 2 or 3 independent donors.

Statistical analysis. Statistical analyses were performed using GraphPad Prism 8 (GraphPad Software, Inc). Student t-test was used to compare two independent groups. Three or more groups were compared with one-way ANOVA test followed by Tukey's multiple comparisons test. p<0.05 was considered to indicate statistical significance.

### 4. Example 4: Results

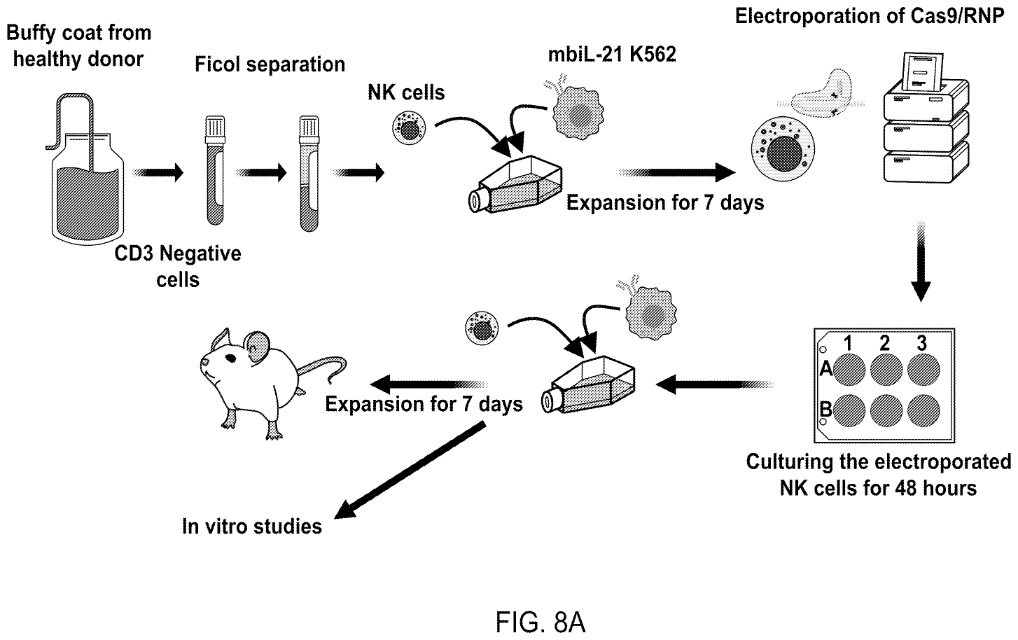

Efficient gene targeting using Cas9/RNP in NK cells. Using Cas9/RNP method, CD38^{KO} NK cells were successfully generated from ex vivo expanded peripheral blood NK (PB-NK) cells of healthy donors (FIG. 8A). Flow cytometric analysis revealed that the CD38 knockout efficiency was 81.9±6.9% (n=5, mean±SD, FIG. 8B). Using magnetic separation, NK cells were purified to over 99% CD38^{KO} to be used for further experiments (FIG. 8C). CD38^{WT} and CD38^{KO} NK cells showed similar expansion rate and the purity of CD38^{KO} NK cells were preserved after subsequent culture (FIG. 8D). No differences was observed in the levels of CD16 expression between CD38^{WT} and CD38^{KO} NK cells (FIG. 7B).

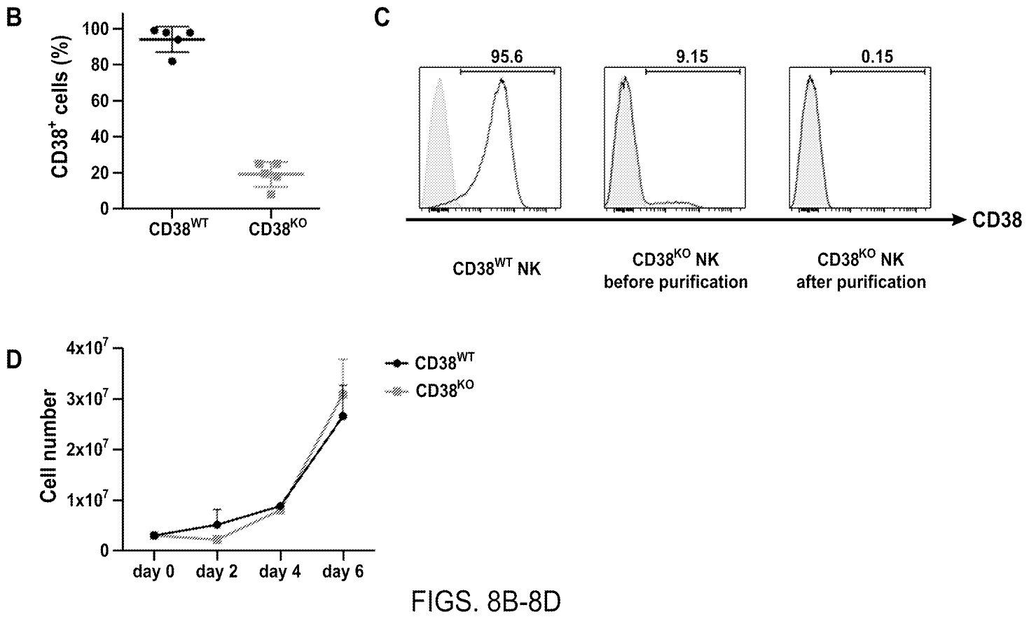

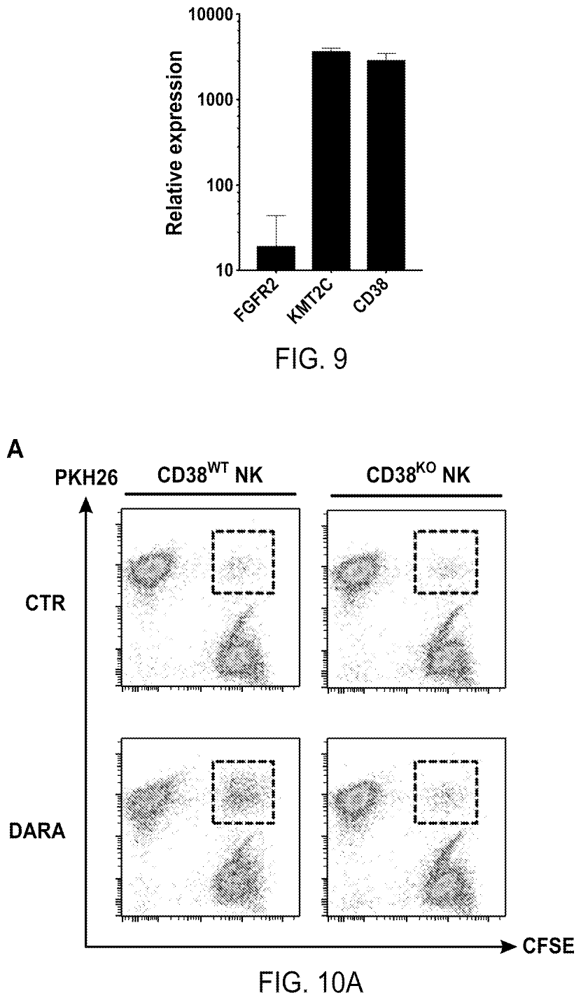

Low off-target effects of Cas9/RNP in NK cells. High-fidelity Cas9 (HiFi-Cas9) has been shown to have low off-target editing due to its rapid degradation after electroporation. To study the off-target effects in CRISPR-modified NK cells, whole genome sequencing (WGS) was performed and 26 genes with SNPs and INDELs exclusive to the CD38^{KO} NK cells were found. Because mutations were restricted to in coding regions, all genes had mutations of moderate or high potential impact. 18 genes had mutations categorized as moderate impact (missense and non-frameshift) and eight genes (including CD38) had mutations categorized as high impact (startloss, stopgain and frameshift) by SnpEff (Table 3). By RNA-seq, only four of the off-target genes with possible high-impact mutations are expressed at meaningful levels in NK cells (CC2D1B, DENND4B, KMT2C, and WDR89, FIG. 9). These results show the efficiency and specificity of this gRNA for CD38 targeting in NK cells.

TABLE 3

| | | | Variant | Variant | | | | | | |
|---|---|---|---|---|---|---|---|---|---|---|
| | | | Allele | Allele | | | | | | |
| | | | Frequency | Frequency | | | | Loc | | |
| | | | In | In | | | | In | | |
| | | | CD38CD | CD38WT | | | | | | |
| Gene | Chr | position | NK | NK | Ref | Alt | Filter | Gene | Effect | Impact |
| ANKR | 2 | 96517484 | 6.90% | 0.00% | T | G | clustered_ | coding_ | Nonsynonymous | MODERATE |
| DB6C | | | | | | | events, | sequence | | |
| | | | | | | | t_lod, | | SNV | |
| | | | | | | | mapping_ | | | |
| | | | | | | | quality | | | |
| APOB | 22 | 39497425 | 19.05% | 0.00% | T | A | PASS | coding_ | Nonsynonymous | MODERATE |
| EC3H | | | | | | | | sequence | SNV | |
| CC2D | 1 | 52825875 | 8.00% | 0.00% | T | TC | clustered_ | coding_ | frameshift | HIGH |
| 1B | | | | | | | events, | sequence | | |
| | | | | | | | t_lod | | | |
| CC2D | 1 | 52825879 | 7.69% | 0.00% | C | CAG | clustered_ | coding_ | frameshift | HIGH |
| 1B | | | | | | | events, | sequence | | |
| | | | | | | | t_lod | | | |
| CC2D | 1 | 52825881 | 7.69% | 0.00% | C | CCTT | clustered_ | coding_ | Frameshift | HIGH |
| 1B | | | | | | GGA | events, | sequence | &stopgain | |
| | | | | | | GTCG | t_lod | | | |
| | | | | | | CAG | | | | |
| | | | | | | CTAG | | | | |
| | | | | | | CCT | | | | |
| | | | | | | CTGT | | | | |
| | | | | | | GAG | | | | |
| | | | | | | G | | | | |
| | | | | | | (SEQ | | | | |
| | | | | | | ID | | | | |
| | | | | | | NO: | | | | |
| | | | | | | 5) | | | | |
| CCDC | 1 | 169388367 | 19.05% | 0.00% | C | T | clustered_ | coding_ | Nonsynonymous | MODERATE |
| 181 | | | | | | | events | sequence | SNV | |
| CCDC | 1 | 169388373 | 18.18% | 0.00% | C | T | clustered_ | coding_ | Nonsynonymous | MODERATE |
| 181 | | | | | | | events, | sequence | | |
| | | | | | | | base_ | | SNV | |
| | | | | | | | quality | | | |
| CD38 | 4 | 15780022 | 12.00% | 0.00% | CCCCG | C | clustered_ | coding_ | startloss& | HIGH |
| | | | | | CCTGG | | events | sequence | nonframeshift | |
| | | | | | AGCCC | | | | deletion | |
| | | | | | TATGG | | | | | |
| | | | | | CCAAC | | | | | |
| | | | | | TGCGA | | | | | |
| | | | | | GTTCA | | | | | |
| | | | | | GCCCG | | | | | |
| | | | | | GTGTC | | | | | |
| | | | | | CGGGG | | | | | |
| | | | | | ACAAA | | | | | |
| | | | | | (SEQ | | | | | |
| | | | | | ID | | | | | |
| | | | | | NO: | | | | | |
| | | | | | 6) | | | | | |
| CD38 | 4 | 15780032 | 31.25% | 0.00% | AGCC | A | clustered_ | coding_ | frameshift& | HIGH |
| | | | | | CTAT | | events | sequence | startloss | |
| | | | | | G | | | | | |
| CD38 | 4 | 15780039 | 13.33% | 0.00% | TG | T | clustered_ | coding_ | frameshift | HIGH |
| | | | | | | | events, | sequence | | |
| | | | | | | | t_lod | | | |
| CD38 | 4 | 15780040 | 12.50% | 0.00% | GGC | G | clustered_ | coding_ | frameshift | HIGH |
| | | | | | CAA | | events | sequence | | |
| CR1 | 1 | 207782852 | 8.00% | 0.00% | T | TTC | clustered_ | coding_ | frameshift | HIGH |
| | | | | | | TGG | events, | sequence | | |
| | | | | | | | t_lod | | | |
| CR1 | 1 | 207782853 | 8.00% | 0.00% | A | AGC | clustered_ | coding_ | frameshift | HIGH |
| | | | | | | TGT | events, | sequence | | |
| | | | | | | GC | t_lod | | | |
| CR1 | 1 | 207782855 | 8.33% | 0.00% | T | TTT | clustered_ | coding_ | frameshift | HIGH |
| | | | | | | ATT | events, | sequence | | |
| | | | | | | AGT | t_lod | | | |
| | | | | | | AG | | | | |
| | | | | | | (SEQ | | | | |
| | | | | | | ID | | | | |
| | | | | | | NO: | | | | |
| | | | | | | 7) | | | | |
| CR1 | 1 | 207782860 | 7.69% | 0.00% | G | A | clustered_ | coding_ | Nonsynonymous | MODERATE |
| | | | | | | | events, | sequence | SNV | |
| | | | | | | | t_lod | | | |
| DENN | 1 | 153903191 | 8.70% | 0.00% | TG | T | clustered_ | coding_ | frameshift | HIGH |
| D4B | | | | | | | events, | sequence | | |
| | | | | | | | t_lod | | | |
| DENN | 1 | 153903197 | 8.33% | 0.00% | CCC | C | clustered_ | coding_ | Nonframeshift | MODERATE |
| D4B | | | | | TAGA | | events, | sequence | deletion | |
| | | | | | GTT | | t_lod | | | |
| | | | | | (SEQ | | | | | |
| | | | | | ID | | | | | |
| | | | | | NO: | | | | | |
| | | | | | 8) | | | | | |
| DENN | 1 | 153903210 | 10.53% | 0.00% | A | T | clustered_ | coding_ | Nonsynonymous | MODERATE |
| D4B | | | | | | | events, | sequence | SNV | |
| | | | | | | | base_ | | | |
| | | | | | | | quality, | | | |
| | | | | | | | read_ | | | |
| | | | | | | | position | | | |
| DENN | 1 | 153903213 | 9.52% | 0.00% | A | T | clustered_ | coding_ | Nonsynonymous | MODERATE |
| D4B | | | | | | | events, | sequence | SNV | |
| | | | | | | | base_ | | | |
| | | | | | | | quality, | | | |
| | | | | | | | read_ | | | |
| | | | | | | | position | | | |
| DENN | 1 | 153903216 | 11.11% | 0.00% | A | AAA | clustered_ | coding_ | stopgain& | HIGH |
| D4B | | | | | | CTC | events, | sequence | nonframeshift | |
| | | | | | | TAG | base_ | | insertion | |
| | | | | | | G | quality, | | | |
| | | | | | | (SEQ | read_ | | | |
| | | | | | | ID | position | | | |
| | | | | | | NO: | | | | |
| | | | | | | 9) | | | | |
| FGF7 | 15 | 49776539 | 12.50% | 0.00% | A | T | PASS | coding_ | Nonsynonymous | MODERATE |
| | | | | | | | | sequence | SNV | |
| FGFR | 10 | 123244918 | 10.53% | 0.00% | G | GGG | PASS | coding_ | Nonframeshift | MODERATE |
| 2 | | | | | | CAC | | sequence | insertion | |
| | | | | | | CGG | | | | |
| | | | | | | CAG | | | | |
| | | | | | | GAA | | | | |
| | | | | | | AGA | | | | |
| | | | | | | CAA | | | | |
| | | | | | | C | | | | |
| | | | | | | (SEQ | | | | |
| | | | | | | ID | | | | |
| | | | | | | NO: | | | | |
| | | | | | | 10) | | | | |
| FGFR | 10 | 123244922 | 10.53% | 0.00% | A | ACCA | PASS | coding_ | frameshift | HIGH |
| 2 | | | | | | ACG | | sequence | | |
| | | | | | | AAC | | | | |
| | | | | | | TGTA | | | | |
| | | | | | | AGG | | | | |
| | | | | | | GCT | | | | |
| | | | | | | (SEQ | | | | |
| | | | | | | ID | | | | |
| | | | | | | NO: | | | | |
| | | | | | | 11) | | | | |
| FHOD1 | 16 | 67270885 | 10.53% | 0.00% | G | A | PASS | coding_ | nonsynonymous | MODERATE |
| | | | | | | | | sequence | SNV | |
| FOXP3 | X | 49107814 | 9.09% | 0.00% | T | A | PASS | coding_ | nonsynonymous | MODERATE |
| | | | | | | | | sequence | SNV | |
| KMT2C | 7 | 151962134 | 9.76% | 0.00% | G | T | PASS | coding_ | stopgain | HIGH |
| | | | | | | | | sequence | | |
| MT- | MT | 9181 | 1.33% | 0.00% | A | G | PASS | coding_ | nonsynonymous | MODERATE |
| ATP6 | | | | | | | | sequence | SNV | |
| MYH4 | 17 | 10352331 | 10.00% | 0.00% | T | G | clustered_ | coding_ | nonsynonymous | MODERATE |
| | | | | | | | events | sequence | SNV | |
| MYH4 | 17 | 10352336 | 8.70% | 0.00% | C | CAGG | clustercd_ | coding_ | nonframeshift | MODERATE |
| | | | | | | | events, | sequence | insertion | |
| | | | | | | | t_ | | | |
| | | | | | | | lod, | | | |
| | | | | | | | read_ | | | |
| | | | | | | | position | | | |
| MYH4 | 17 | 10352338 | 8.70% | 0.00% | T | TGCA | clustered_ | coding_ | nonframeshift | MODERATE |
| | | | | | | GAAGAA | events, | sequence | insertion | |
| | | | | | | (SEQ | base_ | | | |
| | | | | | | ID | quality, | | | |
| | | | | | | NO: | t_ | | | |
| | | | | | | 12) | lod, | | | |
| | | | | | | | read_ | | | |
| | | | | | | | position | | | |
| MYH4 | 17 | 10352340 | 9.09% | 0.00% | C | A | clustered_ | coding_ | nonsynonymous | MODERATE |
| | | | | | | | events, | sequence | SNV | |
| | | | | | | | base_ | | | |
| | | | | | | | quality, | | | |
| | | | | | | | t_ | | | |
| | | | | | | | lod, | | | |
| | | | | | | | read_ | | | |
| | | | | | | | position | | | |
| NBPF14 | 1 | 148252777 | 15.38% | 0.00% | T | TCTC | PASS | coding_ | nonframeshift | MODERATE |
| | | | | | | | | sequence | insertion | |
| OR2T4 | 1 | 248525100 | 12.50% | 0.00% | G | A | clustered_ | coding_ | nonsynonymous | MODERATE |
| | | | | | | | events | sequence | SNV | |
| OR2T4 | 1 | 248525135 | 11.11% | 0.00% | G | A | clustered_ | coding_ | nonsynonymous | MODERATE |
| | | | | | | | events | sequence | SNV | |
| OR2T4 | 1 | 248525138 | 13.79% | 0.00% | C | T | clustered_ | coding_ | nonsynonymous | MODERATE |
| | | | | | | | events | sequence | SNV | |
| PABPC1 | 8 | 101718932 | 7.41% | 0.00% | C | G | clustered_ | coding_ | nonsynonymous | MODERATE |
| | | | | | | | events, | sequence | SNV | |
| | | | | | | | t_lod | | | |
| PABPC1 | 8 | 101718968 | 7.14% | 0.00% | C | T | clustered_ | coding_ | nonsynonymous | MODERATE |
| | | | | | | | events, | sequence | SNV | |
| | | | | | | | t_lod | | | |
| PABPC1 | 8 | 101721705 | 8.33% | 0.00% | G | T | clustered_ | coding_ | nonsynonymous | MODERATE |
| | | | | | | | events, | sequence | SNV | |
| | | | | | | | t_lod | | | |
| PABP | 8 | 101721709 | 8.33% | 0.00% | T | A | clustered_ | coding_ | nonsynonymous | MODERATE |
| C1 | | | | | | | events, | sequence | SNV | |
| | | | | | | | t_ | | | |
| | | | | | | | lod | | | |
| PABP | 8 | 101721812 | 11.54% | 0.00% | G | A | clustered_ | coding_ | nonsynonymous | MODERATE |
| C1 | | | | | | | events | sequence | SNV | |
| PABP | 8 | 101721817 | 10.71% | 0.00% | T | C | clustered_ | coding_ | nonsynonymous | MODERATE |
| C1 | | | | | | | events | sequence | SNV | |
| PABP | 8 | 101721839 | 10.34% | 0.00% | C | A | clustered_ | coding_ | nonsynonymous | MODERATE |
| C1 | | | | | | | events, | sequence | SNV | |
| | | | | | | | t_ | | | |
| | | | | | | | lod | | | |
| FGFR | 10 | 123244918 | 10.53% | 0.00% | G | GGGCACC | PASS | coding_ | nonframeshift | MODERATE |
| 2 | | | | | | GGCAGGA | | sequence | insertion | |
| | | | | | | AAGACAA | | | | |
| | | | | | | C | | | | |
| | | | | | | (SEQ ID | | | | |
| | | | | | | NO: | | | | |
| | | | | | | 10) | | | | |
| FGFR | 10 | 123244922 | 10.53% | 0.00% | A | ACCAACG | PASS | coding_ | frameshift | HIGH |
| 2 | | | | | | AACTGTA | | sequence | | |
| | | | | | | AGGGCT | | | | |
| | | | | | | (SEQ | | | | |
| | | | | | | ID NO: | | | | |
| | | | | | | 11) | | | | |
| PABP | 13 | 25670851 | 9.38% | 0.00% | A | G | PASS | coding_ | nonsynonymous | MODERATE |
| C3 | | | | | | | | sequence | SNV | |
| PABP | 13 | 25671545 | 6.67% | 0.00% | C | A | clustered_ | coding_ | nonsynonymous | MODERATE |
| C3 | | | | | | | events, | sequence | SNV | |
| | | | | | | | t_ | | | |
| | | | | | | | lod | | | |
| PABP | 13 | 25671549 | 9.38% | 0.00% | A | G | clustered_ | coding_ | nonsynonymous | MODERATE |
| C3 | | | | | | | events | sequence | SNV | |
| PABP | 13 | 25671555 | 9.68% | 0.00% | G | A | clustered_ | coding_ | nonsynonymous | MODERATE |
| C3 | | | | | | | events | sequence | SNV | |
| PABP | 13 | 25671558 | 9.38% | 0.00% | C | T | clustered_ | coding_ | nonsynonymous | MODERATE |
| C3 | | | | | | | events | sequence | SNV | |
| PABP | 13 | 25671564 | 10.34% | 0.00% | A | T | clustered_ | coding_ | nonsynonymous | MODERATE |
| C3 | | | | | | | events | sequence | SNV | |
| PABP | 13 | 25671573 | 15.63% | 0.00% | CA | TG | clustered_ | coding_ | nonsynonymous | MODERATE |
| C3 | | | | | | | events | sequence | SNV | |
| PABP | 13 | 25671585 | 18.75% | 0.00% | TA | CC | clustered_ | coding_ | nonsynonymous | MODERATE |
| C3 | | | | | | | events | sequence | SNV | |
| PABP | 13 | 25671699 | 12.50% | 0.00% | GT | CC | clustered_ | coding_ | nonsynonymous | MODERATE |
| C3 | | | | | | | events | sequence | SNV | |
| PABP | 13 | 25671742 | 7.14% | 0.00% | G | A | clustered_ | coding_ | nonsynonymous | MODERATE |
| C3 | | | | | | | events, | sequence | SNV | |
| | | | | | | | t_ | | | |
| | | | | | | | lod | | | |
| PABP | 13 | 25671759 | 7.14% | 0.00% | c | T | clustered_ | coding_ | nonsynonymous | MODERATE |
| C3 | | | | | | | events, | sequence | SNV | |
| | | | | | | | t_ | | | |
| | | | | | | | lod | | | |
| WDR | 14 | 64066367 | 10.00% | 0.00% | A | T | clustered_ | coding_ | nonsynonymous | MODERATE |
| 89 | | | | | | | events, | sequence | SNV | |
| | | | | | | | t_ | | | |
| | | | | | | | lod | | | |
| WDR | 14 | 64066453 | 14.29% | 0.00% | G | A | clustered_ | coding_ | nonsynonymous | MODERATE |
| 89 | | | | | | | events | sequence | SNV | |
| WDR | 14 | 64066471 | 12.00% | 0.00% | GT | AG | clustered_ | coding_ | stopgain | HIGH |
| 89 | | | | | | | events, | sequence | | |
| | | | | | | | mappi | | | |
| | | | | | | | ng_ | | | |
| | | | | | | | quality | | | |
| FGF7 | 15 | 49776539 | 12.50% | 0.00% | A | T | PASS | coding_ | nonsynonymous | MODERATE |
| | | | | | | | | sequence | SNV | |
| FHOD | 16 | 67270885 | 10.53% | 0.00% | G | A | PASS | coding_ | nonsynonymous | MODERATE |
| 1 | | | | | | | | sequence | SNV | |
| MYH | 17 | 10352331 | 10.00% | 0.00% | T | G | clustered_ | coding_ | nonsynonymous | MODERATE |
| 4 | | | | | | | events | sequence | SNV | |
| MYH | 17 | 10352336 | 8.70% | 0.00% | C | CAGG | clustered_ | coding_ | nonframeshift | MODERATE |
| 4 | | | | | | | events, | sequence | insertion | |
| | | | | | | | t_ | | | |
| | | | | | | | lod, | | | |
| | | | | | | | read_ | | | |
| | | | | | | | position | | | |
| MYH | 17 | 10352338 | 8.70% | 0.00% | T | TGCA | clustered_ | coding_ | nonframeshift | MODERATE |
| 4 | | | | | | GAA | events, | sequence | insertion | |
| | | | | | | GAA | t_ | | | |
| | | | | | | (SEQ | lod, | | | |
| | | | | | | ID | read_ | | | |
| | | | | | | NO: | position | | | |
| | | | | | | 12) | | | | |
| MYH | 17 | 10352340 | 9.09% | 0.00% | C | A | clustered_ | coding_ | nonsynonymous | MODERATE |
| 4 | | | | | | | events, | sequence | SNV | |
| | | | | | | | base_ | | | |
| | | | | | | | quality, | | | |
| | | | | | | | t_ | | | |
| | | | | | | | lod, | | | |
| | | | | | | | read_ | | | |
| | | | | | | | position | | | |
| STXB | 17 | 53237239 | 12.50% | 0.00% | T | G | clustered_ | coding_ | nonsynonymous | MODERATE |
| P4 | | | | | | | events, | sequence | SNV | |
| | | | | | | | mapping_ | | | |
| | | | | | | | quality | | | |
| STXB | 17 | 53237241 | 12.50% | 0.00% | G | T | clustered_ | coding_ | nonsynonymous | MODERATE |
| P4 | | | | | | | events, | sequence | SNV | |
| | | | | | | | mapping_ | | | |
| | | | | | | | quality | | | |
| STXB | 17 | 53237250 | 13.04% | 0.00% | A | C | clustered_ | coding_ | nonsynonymous | MODERATE |
| P4 | | | | | | | events, | sequence | SNV | |
| | | | | | | | mapping_ | | | |
| | | | | | | | quality | | | |
| SIGLE | 19 | 51919263 | 11.11% | 0.00% | G | T | clustered_ | coding_ | nonsynonymous | MODERATE |
| C10 | | | | | | | events, | sequence | SNV | |
| | | | | | | | mapping_ | | | |
| | | | | | | | quality | | | |
| SIGLE | 19 | 51919279 | 12.50% | 0.00% | A | G | clustered_ | coding_ | startloss | LOW |
| C10 | | | | | | | events, | sequence | | |
| | | | | | | | mapping_ | | | |
| | | | | | | | quality | | | |
| SIGLE | 19 | 51919302 | 9.52% | 0.00% | T | C | clustered_ | coding_ | nonsynonymous | MODERATE |
| C10 | | | | | | | events, | sequence | SNV | |
| | | | | | | | mapping_ | | | |
| | | | | | | | quality | | | |
| APOB | 22 | 39497425 | 19.05% | 0.00% | T | A | PASS | coding_ | nonsynonymous | MODERATE |
| EC3H | | | | | | | | sequence | SNV | |
| FOXP | X | 49107814 | 9.09% | 0.00% | T | A | PASS | coding_ | nonsynonymous | MODERATE |
| 3 | | | | | | | | sequence | SNV | |
| MT- | MT | 9181 | 1.33% | 0.00% | A | G | PASS | coding_ | nonsynonymous | MODERATE |
| ATP6 | | | | | | | | sequence | SNV | |

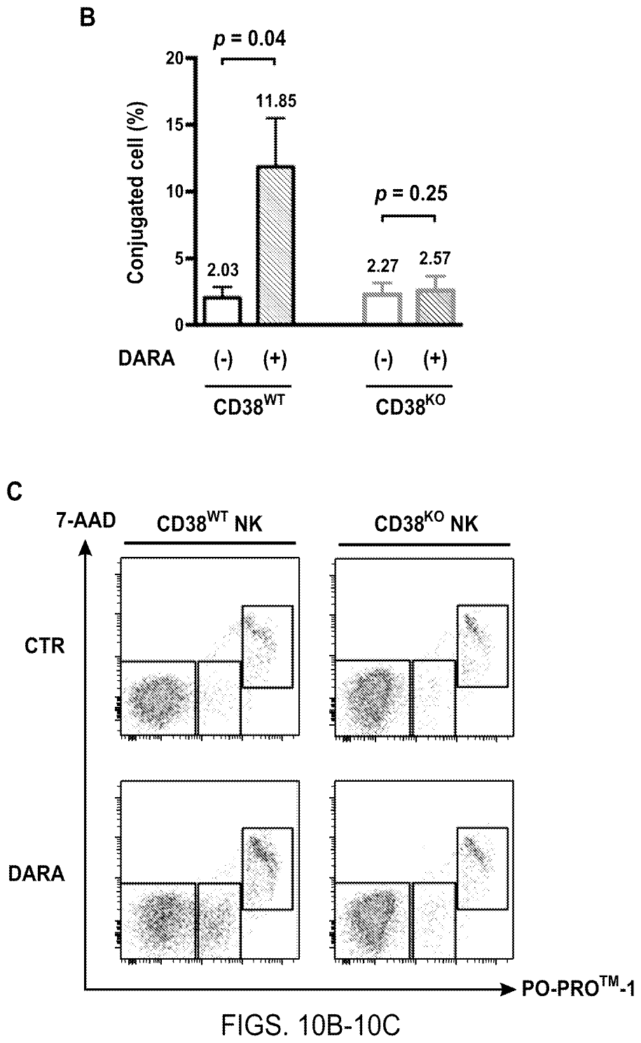

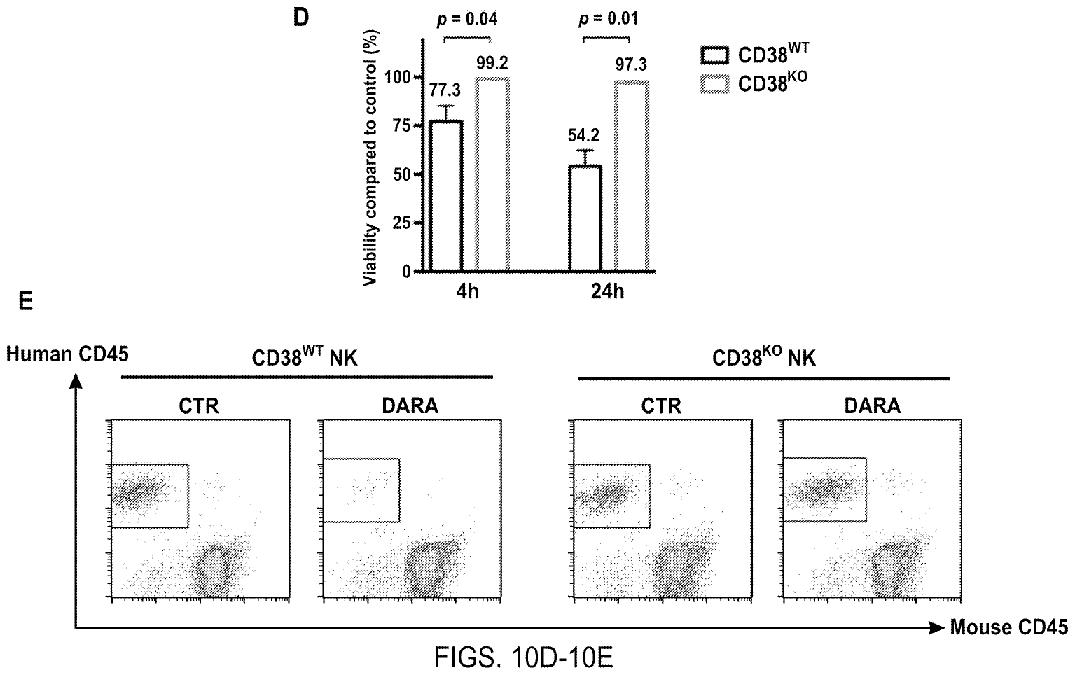

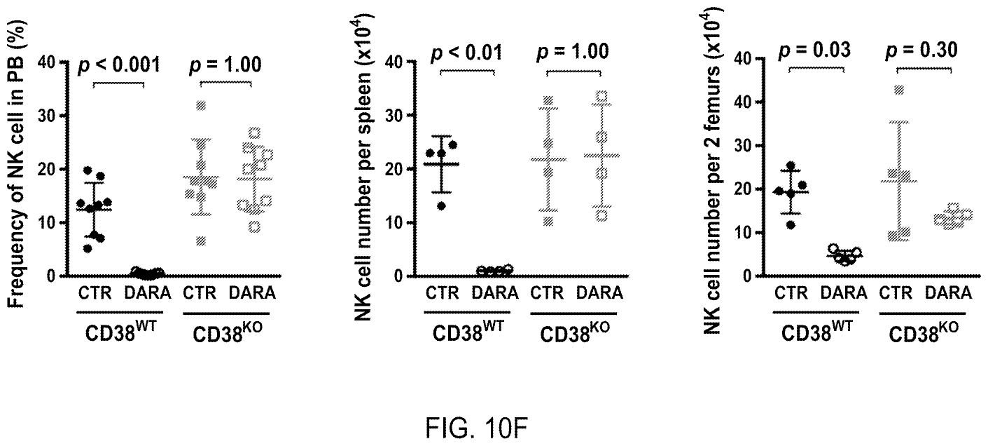

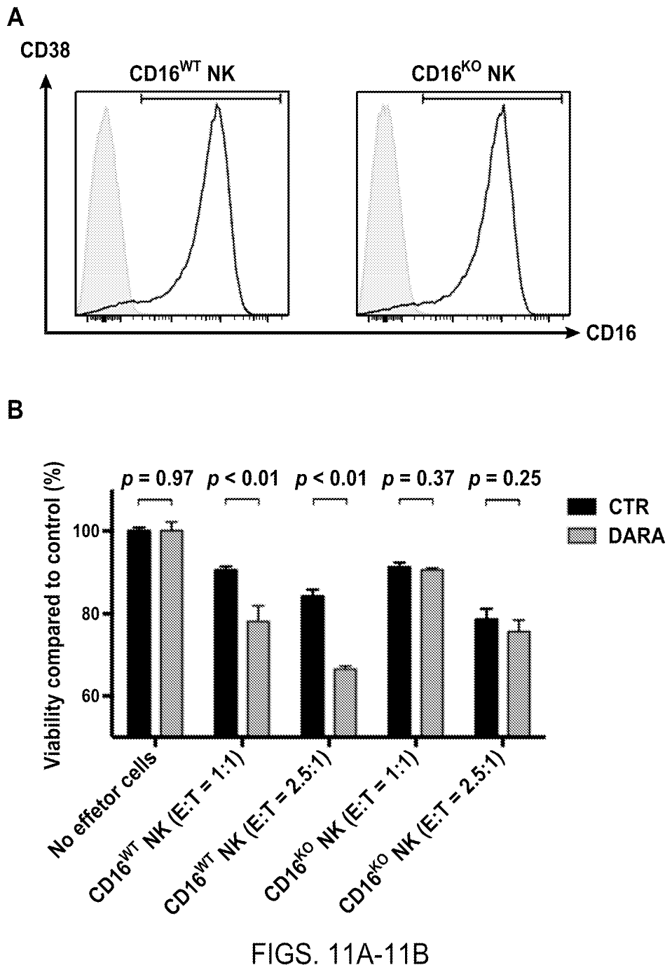

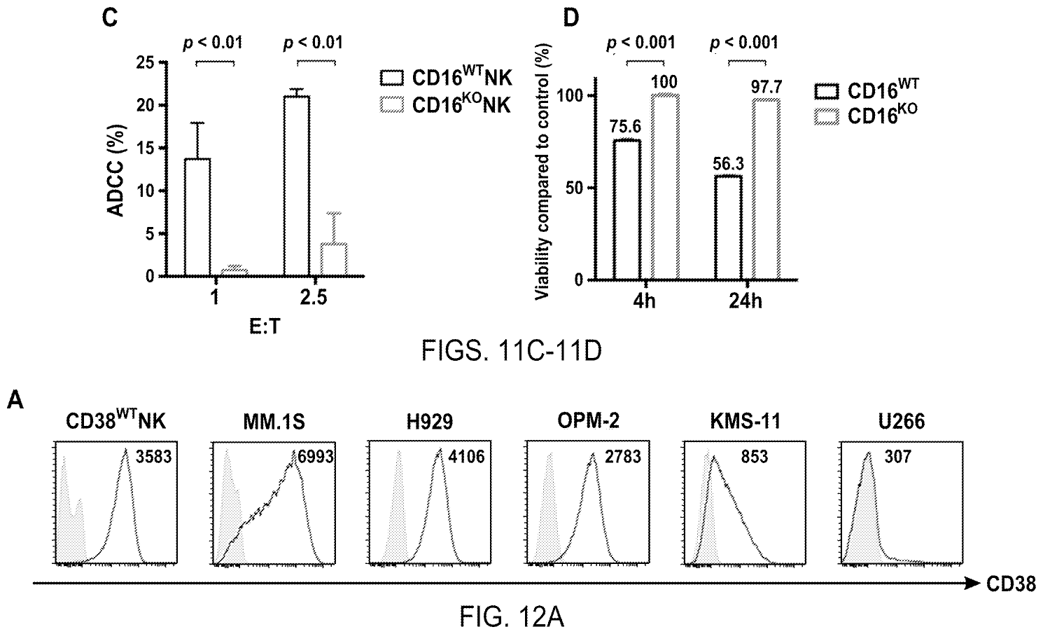

Resistance of CD38^{KO} NK cells to DARA-induced fratricide. DARA induces NK cell fratricide via NK-to-NK ADCC by crosslinking CD38 and CD16. To study if CD38^{KO} NK cells are resistant to DARA-induced fratricide, conjugation and viability of paired CD38^{WT} and CD38^{KO} NK cells were evaluated. DARA increased the formation of CD38^{WT} NK cell conjugates, but did not affect the formation of CD38^{KO} NK cell conjugates (FIGS. 10A-10B). Consistent with this result, DARA induced ADCC-dependent apoptosis of CD38^{WT} NK cells, while CD38^{KO} NK cells preserved their viability throughout the incubation with DARA (FIGS. 10C-10D). Similarly, deletion of CD16 in NK cells also preserved their viability after treatment with DARA. Additionally, no DARA-mediated lysis was observed in the absence of NK cells and complement (FIG. 11B). These results show that CD16 and CD38 are necessary and sufficient for DARA-induced fratricide, and that deletion of CD38 in NK cells renders them resistant to DARA-induced fratricide.

Next, to study if the resistance of CD38^{KO} NK cells to DARA can also contribute to superior persistence in vivo, CD38^{WT} or CD38^{KO} NK cells were fused into NSG mice treated with DARA and examined NK cell frequency in PB, spleen and BM. CD38^{WT} or CD38^{KO} NK cells showed comparable engraftment in control mice (FIGS. 10E-10F). In contrast, treatment with DARA significantly reduced engraftment of CD38^{WT} NK cells but had no effect on persistence of CD38^{KO} NK cells (FIGS. 10E-10F). CD38^{WT} NK cells were depleted by DARA in spleen and BM as well as peripheral blood, while CD38^{KO} NK cells showed no significant depletion in any of these compartments between control and DARA-treated mice (FIG. 10F). Taken together, these results show that CD38^{KO} cells are resistant to DARA in vitro and in vivo.

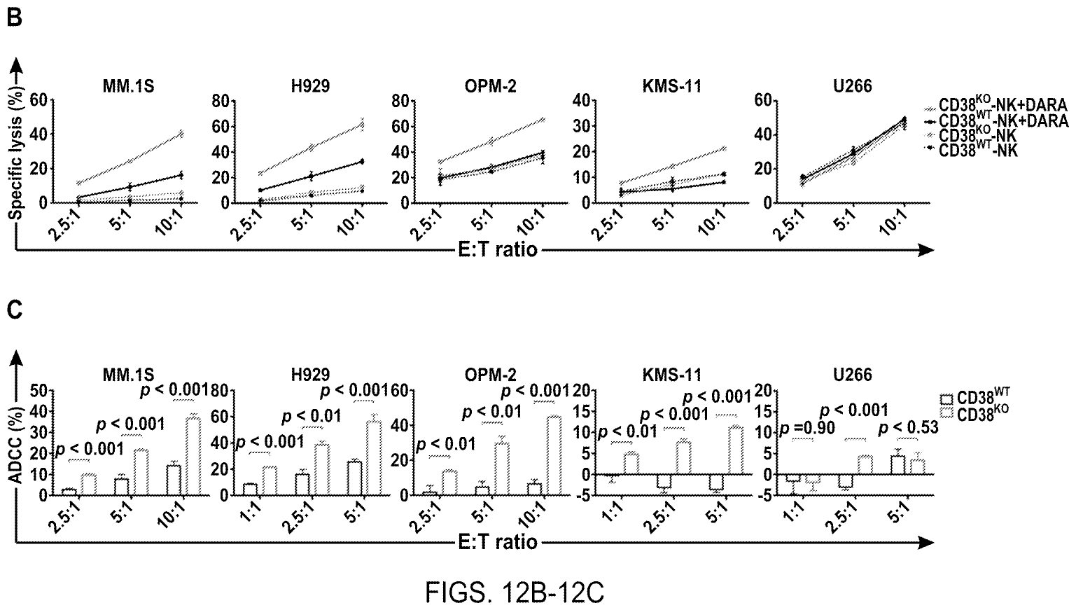

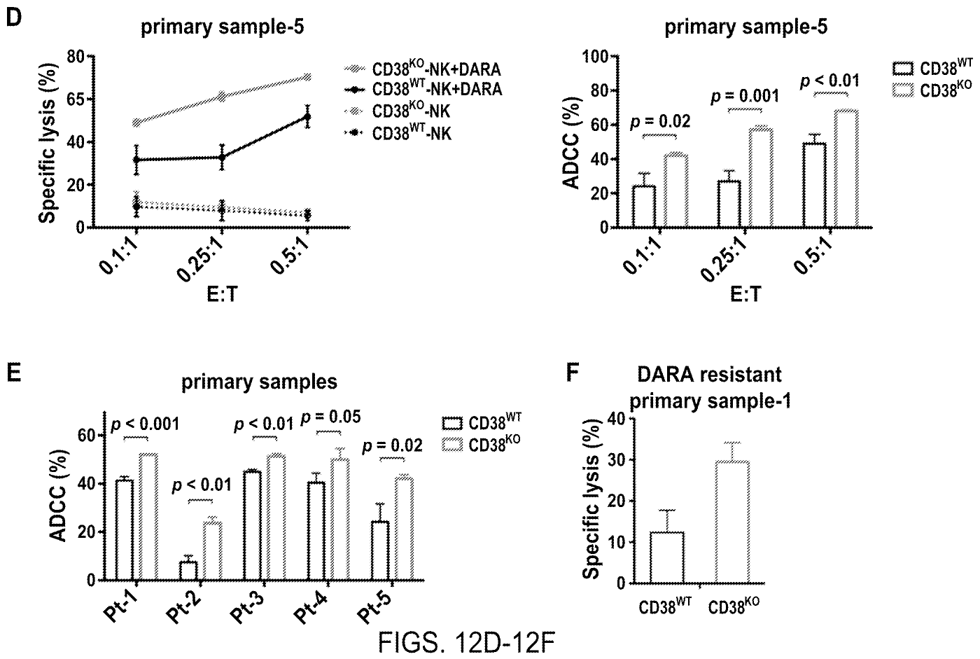

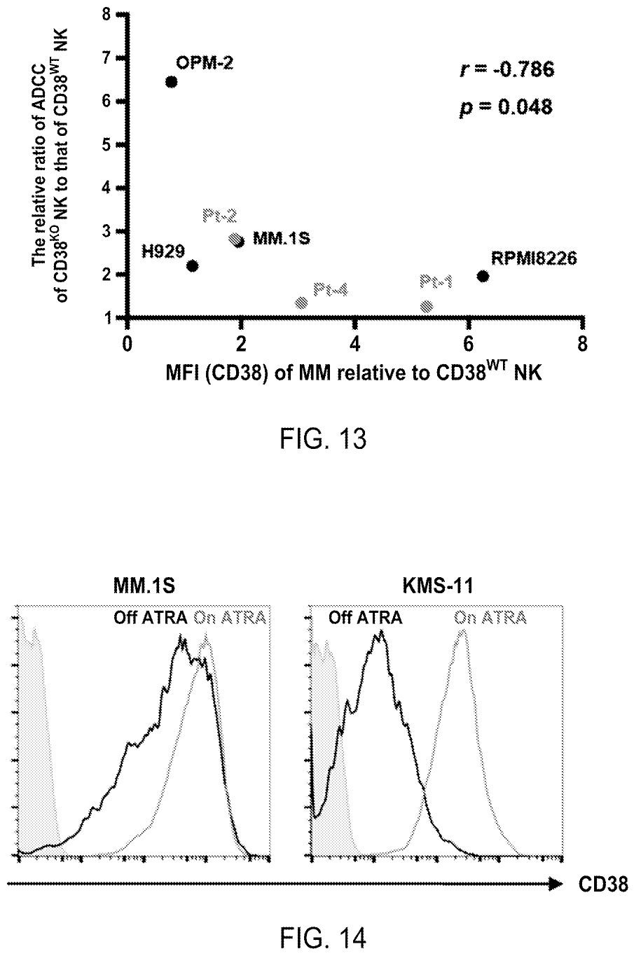

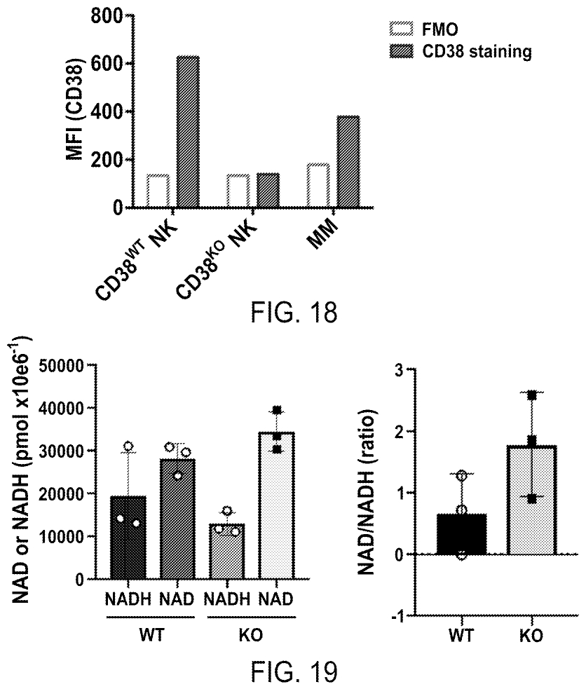

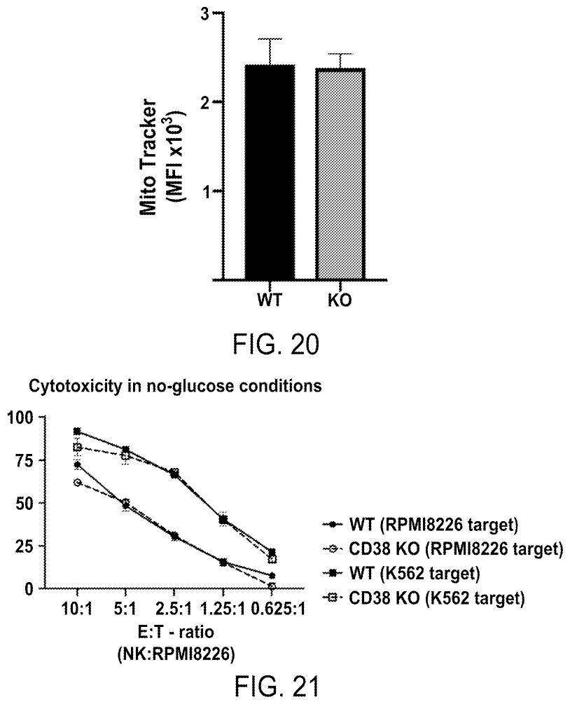

Superior DARA-mediatedADCC of CD38^{KO} NK cells against MM cells. Because DARA-induced depletion of NK cells can blunt cellular dependent cytotoxicity against target cells, CD38^{KO} NK cells can also kill target cells more efficiently than CD38^{WT} NK cells. To study this, the cytotoxicity of paired CD38^{WT} and CD38^{KO} NK cells was tested in the presence or absence of DARA against different MM cell lines with high, low or no levels of CD38 expression (FIG. 12A). The direct cytotoxicity against each MM cell line was equivalent between CD38^{WT} and CD38^{KO} NK cells, however in the presence of DARA, CD38^{KO} NK cells showed significantly higher cytotoxicity against CD38⁺ target cells (FIG. 12B), indicating higher ADCC of CD38^{KO} NK cells (FIG. 12C). Neither CD38^{WT} nor CD38^{KO} NK cells exhibited ADCC against the CD38⁻ cell line U266. CD38^{WT} NK cells showed marginal or no ADCC against MM cells with low levels of CD38 expression such as OPM-2 and KMS-11, whereas CD38^{KO} NK cells demonstrated significantly stronger ADCC against these MM cell lines. Similar to the results with cell lines, CD38^{KO} NK cells showed higher DARA-mediated ADCC activity against primary MM samples (FIGS. 12D-12E), including improved cytotoxicity against primary CD38^{low} MM cells from a DARA-resistant case (FIGS. 12F and 18). The relationship between CD38 expression and the improved ADCC of CD38^{KO} NK compared to CD38^{WT} NK was further studied (FIG. 13) and found a statistically significant inverse correlation (r=−0.786, p=0.048). Altogether, this results indicate that CD38^{KO} NK cells can improve the efficacy of DARA against relapsed and refractory MM that is otherwise DARA-resistant due to low CD38 expression (FIG. 18).

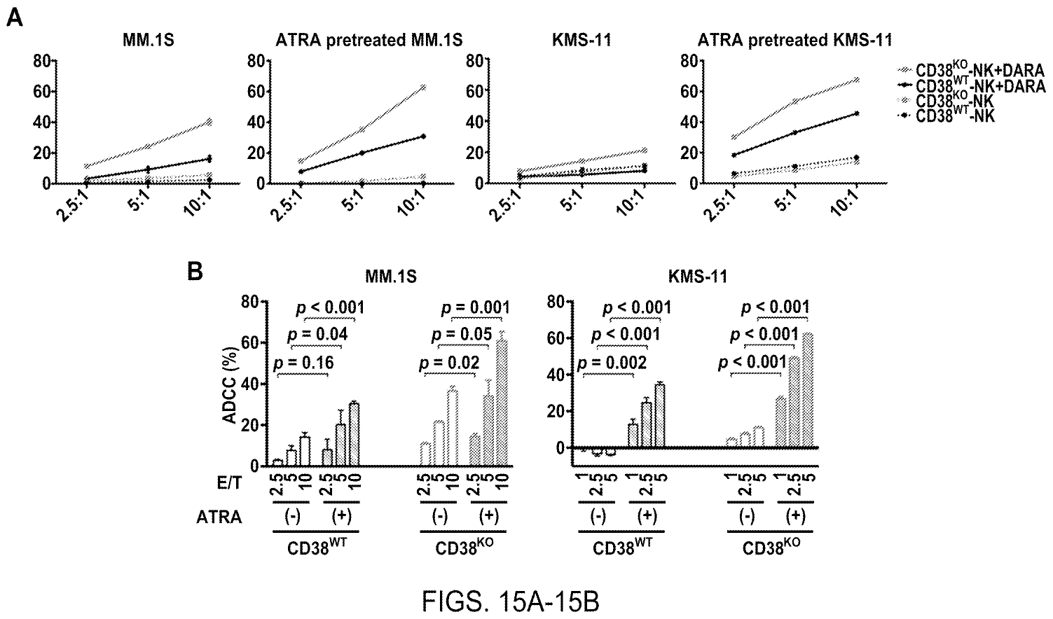

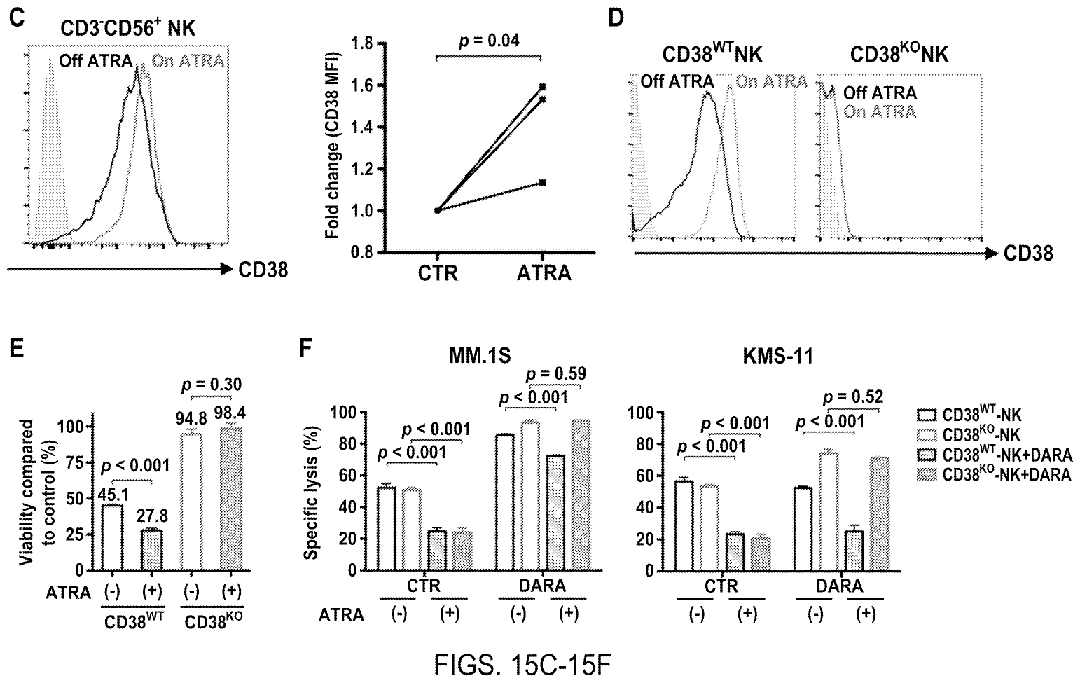

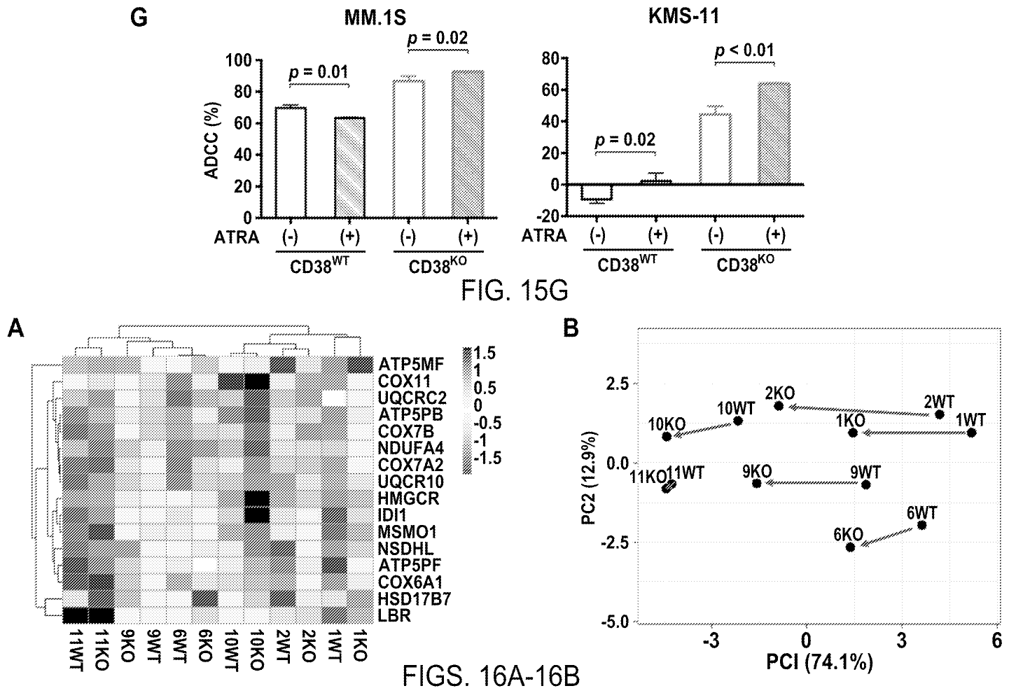

Inhibitory effect of ATRA on NK cell cytotoxicity. Down-regulation of CD38 on MM cells is considered to play an important role in developing resistance to DARA. Treatment with ATRA can overcome this resistance through up-regulation of CD38 levels on CD38^{low} target cells. To investigate synergy in up-regulating CD38 on MM cells and deleting CD38 on NK cells, MM cells were pretreated with ATRA prior to assessing ADCC with DARA and NK cells. It was confirmed that pretreatment with ATRA up-regulates CD38 levels on MM target cells (FIG. 14), and show that it improves DARA-mediated ADCC in the presence of both CD38^{WT} and paired CD38^{KO} NK cells (FIGS. 15A-15B). The direct effects of ATRA on CD38 expression on NK cells in vivo were then examined, using PB-NK of patients with acute promyelocytic leukemia (APL) treated with ATRA during consolidation therapy. Compared to samples obtained prior to treatment, treatment with ATRA was associated with significant up-regulation of CD38 on the PB-NK of these patients (FIG. 15C). Similarly, ex vivo treatment with ATRA up-regulates CD38 levels on CD38^{WT} but not on the paired CD38^{KO} NK cells (FIG. 15D). In addition, ATRA also enhanced fratricide of CD38^{WT} NK cells (FIG. 15E). In contrast to pretreatment of MM cells with ATRA, concurrent treatment of both MM and NK cells with ATRA significantly impaired the DARA-mediated ADCC of CD38^{WT} NK cells but had no impact on the DARA-mediated ADCC of CD38^{KO} NK cells (FIG. 15F). ATRA significantly reduced direct cytotoxicity of both CD38^{WT} and the paired CD38^{KO} NK cells to the same extent (FIG. 15F). Thus, ATRA-induced up-regulation of CD38 on MM target cells may be offset by increased NK cell fratricide and impaired NK cell function, decreasing the overall efficacy of DARA, which can be mitigated by the use of CD38^{KO} NK cells (FIG. 15G).

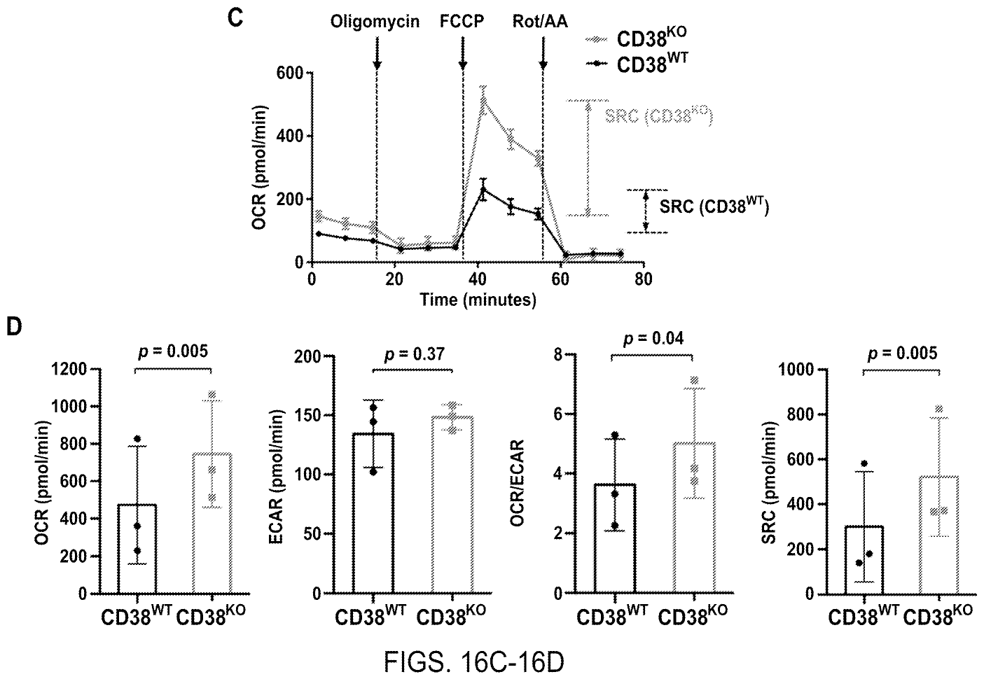

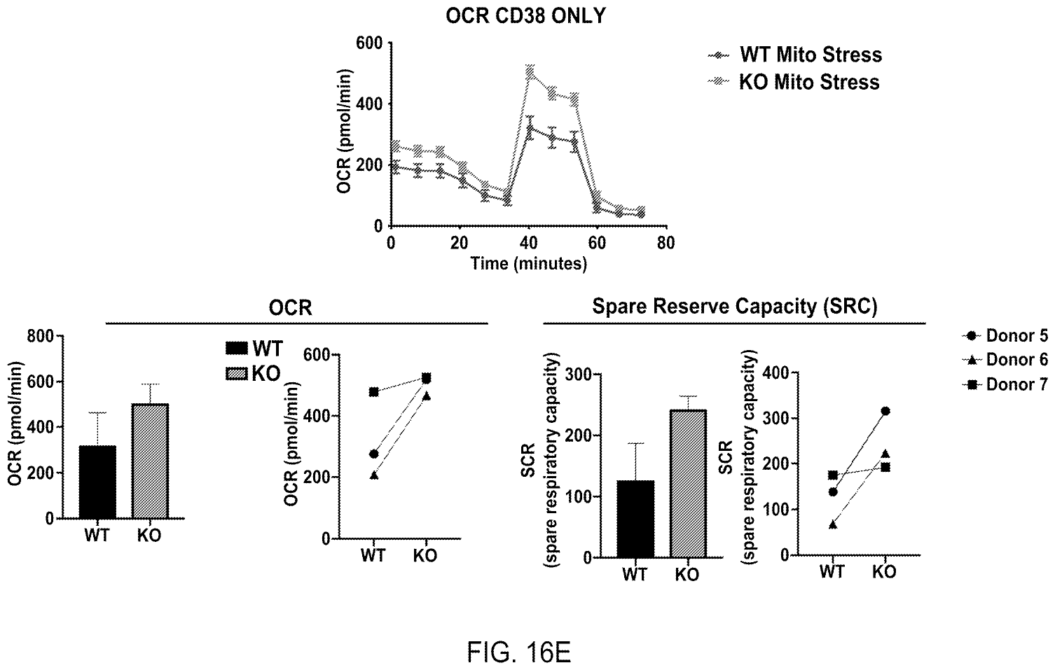

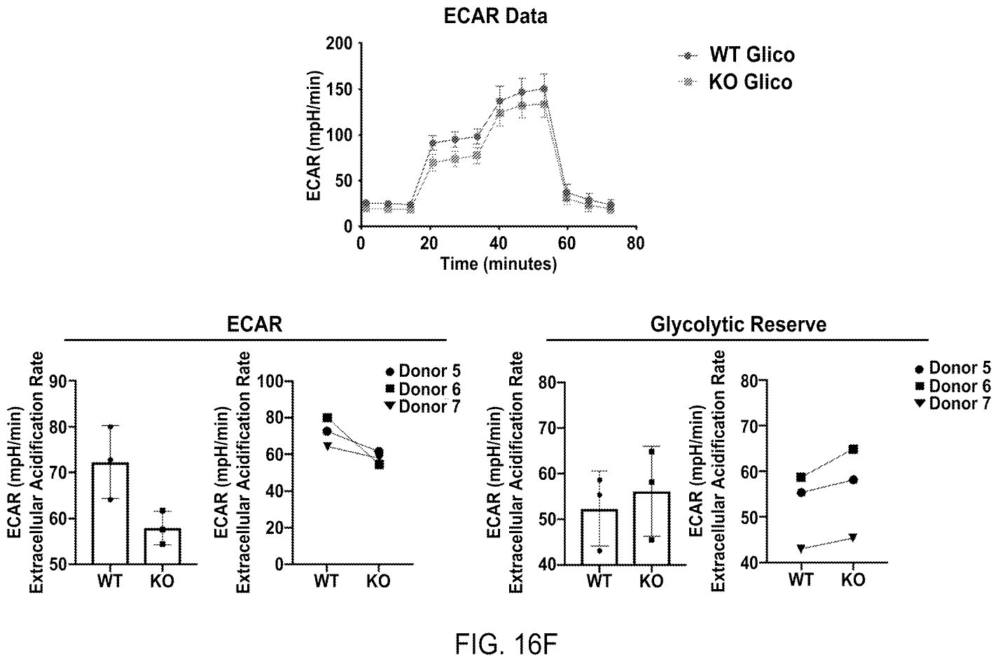

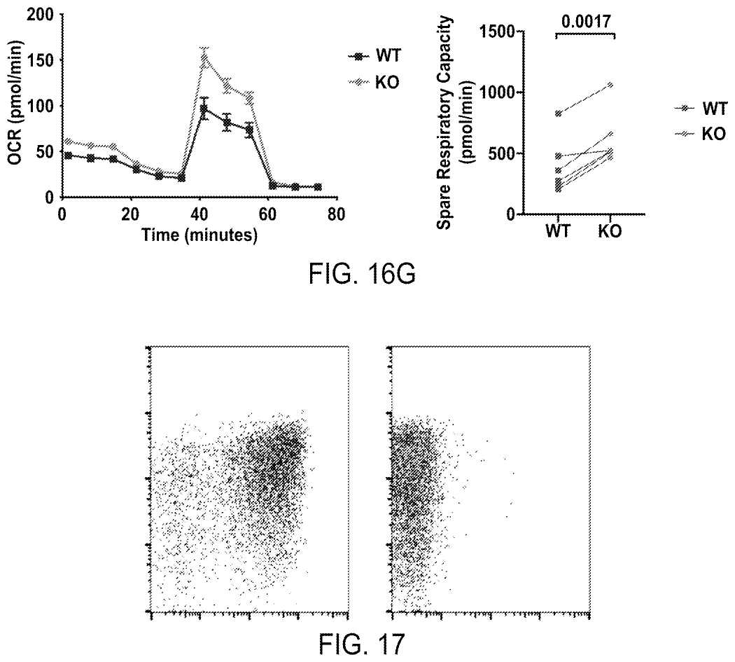

Higher OXPHOS activity in CD38^{KO} NK cells. CD38 is a 46-kDa type II transmembrane glycoprotein and has been shown to have multiple functions including ectoenzymatic activity as a NAD⁺ hydrolase to regulate intracellular NAD⁺ level. Because NAD⁺ is an essential cofactor for enzyme-catalyzed reactions that contributes to ATP production, CD38 plays an important role in cellular metabolism. A recent study reported that CD38 knockout in T cells results in higher levels of intracellular NAD⁺, which fuels OXPHOS and ATP synthesis, and consequently leads to higher cytotoxicity against cancers. Similarly to T-cells, metabolic commitment has a crucial role in cytotoxicity and survival of NK cells in tumor environment. The impact of CD38 knockout on NK cell metabolism was investigated. First, RNA-seq was performed on wild-type and CD38^{KO} NK cells, DEGs were identified, and IPA was used to identify differentially-regulated pathways from the DEGs. IPA showed a significant change in cholesterol biosynthesis (p<0.00001) and OXPHOS (p<0.00001) pathways in CD38^{KO} NK cells. Analysis of genes in those pathways identified a modest but significant increase in expression of mitochondrial genes specifically associated with ATP synthesis, NAD recycling, and electron transport in CD38^{KO} NK cells (Table 1, FIGS. 16A and 17). Principle components analysis revealed significant donor-dependent variation at baseline, but a consistent directional change in response to CD38 deletion among all donor pairs, except for donor #10 which was already at the far end of the spectrum at baseline (FIG. 16B). Considering the up-regulation of genes in these metabolic pathways, the cellular metabolism of CD38^{KO} NK cells was investigated. Using a mitochondrial stress test assay, higher OCR and comparable ECAR were observed, resulting in significantly higher OCR/ECAR ratio in CD38^{KO}NK cells compared to CD38^{WT} NK cells (FIGS. 16C, 16D, 16E, 16F, and 16D). This result indicates that deletion of CD38 induces NK cells to preferentially use OXPHOS to achieve their bioenergetic demands. Importantly, CD38^{KO} NK cells also had higher spare respiratory capacity (SRC) and mitochondrial respiratory capacity compared to CD38^{WT} NK cells (FIG. 16D). These favorable metabolic shifts are consistent with the enhanced DARA-mediated cytotoxicity of CD38^{KO} NK cells.

TABLE 1
Fold changes and p values of DEGs associated with
significantly-altered metabolic pathways identified by IPA.

| | Expression | Expression |
|---|---|---|
| Gene | Fold change | p-value |
| ATP5MF | 1.258 | 0.0323 |
| ATP5PB | 1.132 | 0.0471 |
| ATP5PF | 1.211 | 0.0492 |
| COX11 | 1.129 | 0.00759 |
| COX6A1 | 1.178 | 0.0247 |
| COX7A2 | 1.175 | 0.00928 |
| COX7B | 1.118 | 0.0362 |
| NDUFA4 | 1.173 | 0.0125 |
| UQCR10 | 1.181 | 0.0456 |
| UQCRC2 | 1.144 | 0.0366 |
| HMGCR | 1.089 | 0.0255 |
| HSD17B7 | 1.123 | 0.036 |
| IDI1 | 1.113 | 0.0259 |
| LBR | 1.102 | 0.0362 |
| MSMO1 | 1.124 | 0.00151 |
| NSDH1 | 1.135 | 0.0413 |

The use of DARA has been effective in patients with relapsed/refractory MM. However, despite being incorporated into upfront regimens and its acceptance as standard of care in the treatment of MM, it is increasingly clear that disease relapse is inevitable. Resistance mechanisms of MM cells to treatment with DARA are beginning to be elucidated. Some of these mechanisms overlap with previously proposed mechanisms of resistance to IMiDs or PIs, such as tumor heterogeneity and the role of bone marrow microenvironment, but other mechanisms may be unique to monoclonal antibody therapies.

Monoclonal antibodies eliminate targets through four mechanisms: CDC, ADCC, ADCP and activation-induced cell death through receptor crosslinking. Thus, resistance mechanisms occur through suppression of these mechanisms. For instance, DARA resistance has been associated with over-expression of complement inhibitory proteins (CD55 and CD59) on the surface of MM cells, impairing DARA-mediated CDC. With respect to ADCC, a unique situation occurs with DARA in that NK cells expressing high levels of CD38 are eliminated during treatment, crippling DARA-mediated ADCC. Rescue of DARA-mediated ADCC by adoptive transfer of ex vivo expanded NK cells was successful in a preclinical model using CD38^{low} NK cells. However, these CD38^{low} NK cells reacquired CD38 expression during ex vivo expansion and thus regained susceptibility to DARA-mediated elimination, making clinical translation unlikely. In addition, patients with multiple myeloma—particularly those treated with DARA—have relatively low numbers of immune cells, including NK cells. Thus, we focused our studies on allogeneic NK cells in which a third-party universal-donor strategy would allow the selection of donors with desirable KIR genotypes or FCGR3A polymorphisms for optimal NK cell function.

Here, CD38^{KO} NK cells were generated using CRISPR/Cas9 system. These cells were resistant to DARA-induced conjugation and fratricide and persisted in the presence of DARA in vivo. CD38^{KO} NK cells showed superior ADCC activity against MM cell lines and primary samples when compared to the paired CD38^{WT} cells. CD38^{KO} NK cells were particularly effective against MM cell lines with low CD38 expression and MM cells from a patient who relapsed during DARA treatment, whereas CD38^{WT} NK cells had minimal to no activity against those target cells. Given the selective pressure for low CD38-expressing MM cells during treatment with DARA, CD38^{KO} NK cells can reinforce the therapeutic effect of DARA against this residual disease.

Deletion of CD38 on NK cells was associated with increased mitochondrial respiratory capacity of these cells and a compensatory transcriptomic profile favoring OXPHOS metabolism and cholesterol synthesis. Although the role of this metabolic shift in effector function of CD38^{KO} NK cells was not directly investigated, previous studies showed that knocking out CD38 in T cells results in higher OXPHOS activity and anti-tumor effect.

Antigen density is an important factor in target recognition and effector function by monoclonal antibodies and thus up-regulation of CD38 levels on target cells has the potential to enhance DARA activity. Recently, the clinical synergy between IMiDs and DARA has been attributed in part to the up-regulation of CD38 on MM cells by IMiDs. ATRA was also shown to up-regulate CD38 levels on MM cells and sensitize them to DARA-mediated CDC and ADCC. As opposed to IMiDs that have a known favorable immunomodulatory profile, little is known about the effects of ATRA on the ADCC activity of NK cells. Here, it was confirmed that treatment with ATRA up-regulates CD38 levels on MM cells but also showed that it up-regulates CD38 on ex vivo expanded NK cells and on in vivo circulating NK cells of patients with APL. This modulatory effect on NK cells may enhance DARA-induced fratricide and impair DARA-mediated ADCC against MM cells in vivo. Although CD38^{KO}NK cells were free from fratricide, direct cytotoxicity of both CD38^{WT} and CD38^{KO} NK cell was significantly suppressed by ATRA. Thus, ATRA-mediated up-regulation of CD38 levels on MM cells was offset by its negative impact on NK cell function. Taken together, the net result of treatment with ATRA was overall impaired DARA-mediated ADCC activity of CD38^{WT} NK cells, while the use of CD38^{KO} NK cells rescued the negative impact of ATRA on ADCC.

It is important to mention that treatment with ATRA can boost DARA-mediated CDC via decreasing CD55 and CD59 expression on MM cells. Even though most monoclonal antibodies for clinical development are chosen based on their CDC activity, it is unclear which of the four mechanisms of action plays the most important role in the clinical settings. Preclinical models to differentiate these mechanisms have been difficult because of the high efficacy of DARA alone in eradicating MM in murine xenografts, such that the addition of ex vivo expanded NK cells has a relatively small benefit over DARA alone. Development of CD38^{lo} MM patient-derived xenografts may be useful for pharmacokinetic modeling of DARA and NK cell combinations and in comparing CD38^{KO} and CD38^{WT} NK cells. The current clinical trial (NCT02751255) testing the combination of ATRA and DARA for patients with MM uses a unique therapeutic schedule that provides transient and staggered exposure to these drugs. DARA pharmacokinetics is relatively constant during treatment, and ATRA levels are strictly regulated by complex systemic and tissue dependent feedback mechanisms. In addition to the clinical outcomes using this combination, it is of interest to understand the impact of this treatment schedule on NK cells numbers, functions and DARA-mediated ADCC and CDC. Additionally, It has been shown that anti-KIR antibodies enhance daratumumab-mediated lysis of primary myeloma cells. Investigating the impact of KIR function/genotype and FCRGIIIA polymorphisms on the ADCC activity of CD38^{KO} and CD38^{WT} NK cells is an important area of future research and a necessary step towards clinical translation of these findings.

This study provided proof of concept that CD38^{KO} NK cells can boost the effects of DARA against MM cells. The newly developed DNA-free approach using Cas9/RNP succeeded in efficient generation of CD38^{KO} NK cells with very low frequency of off-target effects as assessed by WGS.

Methods were also applied here to expanding genetically-modified NK cells, which is critical to achieve clinically significant numbers. Collectively, these results demonstrate the rationality and feasibility of ex vivo expanded CD38^{KO} NK cells for adoptive immune therapy in combination with DARA against MM but also potentially other CD38⁺ hematologic malignancies such as acute myeloid leukemia, and B and T cell lymphoblastic leukemias and lymphomas. Additionally, the method for CD38 deletion in NK cells can be applied to generate CD38 chimeric antigen receptor-NK cells to avoid their fratricide during cell manufacturing and effective antitumor activity in vivo.

### 5. Example 5: Combination Therapy Using and Isatuximab

Recognizing the efficacy of the NK CD38^{KO} NK cells for adoptive immune therapy in combination with DARA against MM, other anti-CD38 immunotherapies, other than DARA, such as isatuximab, can be utilized in the treatment of cancer.

Resistance of CD38^{KO} NK cells to isatuximab-induced fratricide. Like DARA, Isatuximab induces NK cell fratricide via NK-to-NK ADCC by crosslinking CD38 and CD16. To study if CD38^{KO} NK cells are resistant to isatuximab-induced fratricide, conjugation and viability of paired CD38^{WT} and CD38^{KO} NK cells can be evaluated. Isatuximab can increase the formation of CD38^{WT} NK cell conjugates, but not affect the formation of CD38^{KO} NK cell conjugates. Also, isatuximab can induce ADCC-dependent apoptosis of CD38^{WT} NK cells, while CD38^{KO} NK cells preserve their viability throughout the incubation with isatuximab. Similarly, deletion of CD16 in NK cells also preserve their viability after treatment with isatuximab. These results show that CD16 and CD38 are necessary and sufficient for isatuximab-induced fratricide, and that deletion of CD38 in NK cells renders them resistant to isatuximab-induced fratricide.

Next, to study if the resistance of CD38^{KO} NK cells to isatuximab can also contribute to superior persistence in vivo, CD38^{WT} or CD38^{KO} NK cells can be fused into NSG mice treated with isatuximab and NK cell frequency in PB, spleen and BM. CD38^{WT} or CD38^{KO} can be examined. Treatment with isatuximab can significantly reduce engraftment of CD38^{WT}NK cells but had no effect on persistence of CD38^{KO} NK cells. As a result of the treatment, CD38^{WT} NK cells are depleted by isatuximab in spleen and BM as well as peripheral blood, while CD38^{KO} NK cells show no significant depletion in any of these compartments between control and isatuximab-treated mice.

Superior isatuximab-mediated ADCC of CD38^{KO} NK cells against MM cells. Because DARA-induced depletion of NK cells can blunt cellular dependent cytotoxicity against target cells, CD38^{KO} NK cells can also kill target cells more efficiently than CD38^{WT} NK cells. To study if this also applies to isatuximab, the cytotoxicity of paired CD38^{WT} and CD38^{KO} NK cells can be tested in the presence or absence of isatuximab against different MM cell lines with high, low or no levels of CD38 expression. The direct cytotoxicity against each MM cell line is equivalent between CD38^{WT} and CD38^{KO} NK cells, however in the presence of isatuximab, CD38^{KO} NK cells show significantly higher cytotoxicity against CD38⁺ target cells, indicating higher ADCC of CD38^{KO} NK cells. As in the DARA experiments, CD38^{WT} NK cells show marginal or no ADCC against MM cells with low levels of CD38 expression such as OPM-2 and KMS-11, whereas CD38^{KO} NK cells demonstrate significantly stronger ADCC against these MM cell lines. Similar to the results with cell lines, CD38^{KO} NK cells show higher isatuximab-mediated ADCC activity against primary MM samples

### E. REFERENCES

Abdallah N, Kumar S K. Daratumumab in untreated newly diagnosed multiple myeloma. Ther Adv Hematol. 2019; 10:2040620719894871.
Alici, E., Sutlu, T. & Sirac Dilber, M. Retroviral gene transfer into primary human natural killer cells. Methods Mol Biol. 506 127-137, doi:10.1007/978-1-59745-409-4_10, (2009).
Alonso S, Hernandez D, Chang Y T, et al. Hedgehog and retinoid signaling alters multiple myeloma microenvironment and generates bortezomib resistance. J Clin Invest. 2016; 126(12):4460-4468.
Baertsch M A, Hundemer M, Hillengass J, Goldschmidt H, Raab M S. Therapeutic monoclonal antibodies in combination with pomalidomide can overcome refractoriness to both agents in multiple myeloma: A case-based approach. Hematol Oncol. 2018; 36(1):258-261.
Bhatnagar V, Gormley N J, Luo L, et al. FDA Approval Summary: Daratumumab for Treatment of Multiple Myeloma After One Prior Therapy. Oncologist. 2017; 22(11):1347-1353.
Bride K L, Vincent T L, Im S Y, et al. Preclinical efficacy of daratumumab in T-cell acute lymphoblastic leukemia. Blood. 2018; 131(9):995-999.
Carlsten, M. & Childs, R. W. Genetic Manipulation of NK Cells for Cancer Immunotherapy: Techniques and Clinical Implications. Front Immunol. 6 266, doi:10.3389/fimmu.2015.00266, (2015).
Casneuf T, Xu X S, Adams H C, 3rd, et al. Effects of daratumumab on natural killer cells and impact on clinical outcomes in relapsed or refractory multiple myeloma. Blood Adv. 2017; 1(23):2105-2114.
Chatterjee S, Daenthanasanmak A, Chakraborty P, et al. CD38-NAD(+)Axis Regulates Immunotherapeutic Anti-Tumor T Cell Response. Cell Metab. 2018; 27(1):85-100 e108.
Chini E N. CD38 as a regulator of cellular NAD: a novel potential pharmacological target for metabolic conditions. Curr Pharm Des. 2009; 15(1):57-63.
Cingolani P, Platts A, Wang le L, et al. A program for annotating and predicting the effects of single nucleotide polymorphisms, SnpEff: SNPs in the genome of Drosophila melanogaster strain w1118; iso-2; iso-3. Fly (Austin). 2012; 6(2):80-92.
Ciurea S O, Schafer J R, Bassett R, et al. Phase 1 clinical trial using mbIL21 ex vivo-expanded donor-derived NK cells after haploidentical transplantation. Blood. 2017; 130(16):1857-1868.
Clemens P L, Yan X, Lokhorst H M, et al. Pharmacokinetics of Daratumumab Following Intravenous Infusion in Relapsed or Refractory Multiple Myeloma After Prior Proteasome Inhibitor and Immunomodulatory Drug Treatment. Clin Pharmacokinet. 2017; 56(8):915-924.
de Haart S J, Holthof L, Noort W A, et al. Sepantronium bromide (YM155) improves daratumumab-mediated cellular lysis of multiple myeloma cells by abrogation of bone marrow stromal cell-induced resistance. Haematologica. 2016; 101(8):e339-342.
de Weers M, Tai Y T, van der Veer M S, et al. Daratumumab, a novel therapeutic human CD38 monoclonal antibody, induces killing of multiple myeloma and other hematological tumors. J Immunol. 2011; 186(3):1840-1848.
Denman C J, Senyukov V V, Somanchi S S, et al. Membrane-bound IL-21 promotes sustained ex vivo proliferation of human natural killer cells. PLoS One. 2012; 7(1):e30264.
DeWitt, M. A., Corn, J. E. & Carroll, D. Genome editing via delivery of Cas9 ribonucleoprotein. Methods. 121-122 9-15, (2017).
Di Gaetano N, Cittera E, Nota R, et al. Complement activation determines the therapeutic activity of rituximab in vivo. J Immunol. 2003; 171(3):1581-1587.
Dimopoulos M A, Oriol A, Nahi H, et al. Daratumumab, Lenalidomide, and Dexamethasone for Multiple Myeloma. N Engl J Med. 2016; 375(14):1319-1331.
Eyquem, J. et al. Targeting a CAR to the TRAC locus with CRISPR/Cas9 enhances tumour rejection. Nature. 543 (7643), 113-117, doi:10.1038/nature21405, (2017).
Fedele P L, Willis S N, Liao Y, et al. IMiDs prime myeloma cells for daratumumab-mediated cytotoxicity through loss of Ikaros and Aiolos. Blood. 2018; 132(20):2166-2178.
Gandhi U H, Cornell R F, Lakshman A, et al. Outcomes of patients with multiple myeloma refractory to CD38-targeted monoclonal antibody therapy. Leukemia. 2019; 33(9):2266-2275.
Gao Z, Tong C, Wang Y, Chen D, Wu Z, Han W. Blocking CD38-driven fratricide among T cells enables effective antitumor activity by CD38-specific chimeric antigen receptor T cells. J Genet Genomics. 2019; 46(8):367-377.
Gavriatopoulou M, Kastritis E, Ntanasis-Stathopoulos I, et al. The addition of IMiDs for patients with daratumumab-refractory multiple myeloma can overcome refractoriness to both agents. Blood. 2018; 131(4):464-467.
Guo Y, Feng K, Tong C, et al. Efficiency and side effects of anti-CD38 CAR T cells in an adult patient with relapsed B-ALL after failure of bi-specific CD19/CD22 CAR T cell treatment. Cell Mol Immunol. 2020.
Guven, H. et al. Efficient gene transfer into primary human natural killer cells by retroviral transduction. Exp Hematol. 33 (11), 1320-1328, doi:10.1016/j.exphem.2005.07.006, (2005).
Hari P, Raj R V, Olteanu H. Targeting CD38 in Refractory Extranodal Natural Killer Cell-T-Cell Lymphoma. N Engl J Med. 2016; 375(15):1501-1502.
Howard M, Grimaldi J C, Bazan J F, et al. Formation and hydrolysis of cyclic ADP-ribose catalyzed by lymphocyte antigen CD38. Science. 1993; 262(5136):1056-1059.
Hu Y, Turner M J, Shields J, et al. Investigation of the mechanism of action of alemtuzumab in a human CD52 transgenic mouse model. Immunology. 2009; 128(2):260-270.
Hu Y, Wang H, Wang Q, Deng H. Overexpression of CD38 decreases cellular NAD levels and alters the expression of proteins involved in energy metabolism and antioxidant defense. J Proteome Res. 2014; 13(2):786-795.
Kararoudi M N, Likhite S, Elmas E, Schwartz M, Meyer K, Lee D A. Highly efficient site-directed gene insertion in primary human natural killer cells using homologous recombination and CRISPaint delivered by AAV. bioRxiv. 2019:743377.
Kelly B J, Fitch J R, Hu Y, et al. Churchill: an ultra-fast, deterministic, highly scalable and balanced parallelization strategy for the discovery of human genetic variation in clinical and population-scale genomics. Genome Biol. 2015; 16:6.
Kim, S., Kim, D., Cho, S. W., Kim, J. & Kim, J. S. Highly efficient RNA-guided genome editing in human cells via delivery of purified Cas9 ribonucleoproteins. Genome Res. 24 (6), 1012-1019, doi:10.1101/gr.171322.113, (2014).
Kishimoto H, Hoshino S, Ohori M, et al. Molecular mechanism of human CD38 gene expression by retinoic acid. Identification of retinoic acid response element in the first intron. J Biol Chem. 1998; 273(25):15429-15434.
Krejcik J, Casneuf T, Nijhof I S, et al. Daratumumab depletes CD38+ immune regulatory cells, promotes T-cell expansion, and skews T-cell repertoire in multiple myeloma. Blood. 2016; 128(3):384-394.
Krejcik J, Frerichs K A, Nijhof I S, et al. Monocytes and Granulocytes Reduce CD38 Expression Levels on Myeloma Cells in Patients Treated with Daratumumab. Clin Cancer Res. 2017; 23(24):7498-7511.
Kumar S K, Dimopoulos M A, Kastritis E, et al. Natural history of relapsed myeloma, refractory to immunomodulatory drugs and proteasome inhibitors: a multicenter IMWG study. Leukemia. 2017; 31(11):2443-2448.
Laubach J, Garderet L, Mahindra A, et al. Management of relapsed multiple myeloma: recommendations of the International Myeloma Working Group. Leukemia. 2016; 30(5):1005-1017.
Lee, D. A., Verneris, M. R. & Campana, D. Acquisition, preparation, and functional assessment of human NK cells for adoptive immunotherapy. Methods Mol Biol. 651 61-77, doi:10.1007/978-1-60761-786-0_4, (2010).
Leone R D, Emens L A. Targeting adenosine for cancer immunotherapy. J Immunother Cancer. 2018; 6(1):57.
Leung-Hagesteijn C, Erdmann N, Cheung G, et al. Xbp1s-negative tumor B cells and pre-plasmablasts mediate therapeutic proteasome inhibitor resistance in multiple myeloma. Cancer Cell. 2013; 24(3):289-304.
Li A, He M, Wang H, et al. All-trans retinoic acid negatively regulates cytotoxic activities of nature killer cell line 92. Biochem Biophys Res Commun. 2007; 352(1):42-47.
Liang, X. et al. Rapid and highly efficient mammalian cell engineering via Cas9 protein transfection. J Biotechnol. 208 44-53, doi:10.1016/j.jbiotec.2015.04.024, (2015).
Liang, Z. et al. Efficient DNA-free genome editing of bread wheat using CRISPR/Cas9 ribonucleoprotein complexes. Nat Commun. 8 14261, doi:10.1038/ncomms14261, (2017).
Liu J, Zhao Y J, Li W H, et al. Cytosolic interaction of type III human CD38 with CIB1 modulates cellular cyclic ADP-ribose levels. Proc Natl Acad Sci USA. 2017.
Maloney D G, Grillo-Lopez A J, White C A, et al. IDEC-C2B8 (Rituximab) anti-CD20 monoclonal antibody therapy in patients with relapsed low-grade non-Hodgkin's lymphoma. Blood. 1997; 90(6):2188-2195.
Martin T, Baz R, Benson D M, et al. A phase 1b study of isatuximab plus lenalidomide and dexamethasone for relapsed/refractory multiple myeloma. Blood. 2017; 129(25):3294-3303.
Mateos M V, Dimopoulos M A, Cavo M, et al. Daratumumab plus Bortezomib, Melphalan, and Prednisone for Untreated Myeloma. N Engl J Med. 2018; 378(6):518-528.
Mehta, R. S. & Rezvani, K. Chimeric Antigen Receptor Expressing Natural Killer Cells for the Immunotherapy of Cancer. Front Immunol. 9 283, doi:10.3389/fimmu.2018.00283, (2018).
Meyer S, Leusen J H, Boross P. Regulation of complement and modulation of its activity in monoclonal antibody therapy of cancer. MAbs. 2014; 6(5):1133-1144.
Minard-Colin V, Xiu Y, Poe J C, et al. Lymphoma depletion during CD20 immunotherapy in mice is mediated by macrophage FcgammaRI, FcgammaRIII, and FcgammaRIV. Blood. 2008; 112(4):1205-1213.
Moreau P, Attal M, Hulin C, et al. Bortezomib, thalidomide, and dexamethasone with or without daratumumab before and after autologous stem-cell transplantation for newly diagnosed multiple myeloma (CASSIOPEIA): a randomised, open-label, phase 3 study. Lancet. 2019; 394(10192):29-38.
Naeimi Kararoudi M, Dolatshad H, Trikha P, et al. Generation of Knock-out Primary and Expanded Human NK Cells Using Cas9 Ribonucleoproteins. J Vis Exp. 2018(136).
Naik J, Themeli M, de Jong-Korlaar R, et al. CD38 as a therapeutic target for adult acute myeloid leukemia and T-cell acute lymphoblastic leukemia. Haematologica. 2019; 104(3):e100-e103.
Nijhof I S, Casneuf T, van Velzen J, et al. CD38 expression and complement inhibitors affect response and resistance to daratumumab therapy in myeloma. Blood. 2016; 128(7):959-970.
Nijhof I S, Groen R W, Lokhorst H M, et al. Upregulation of CD38 expression on multiple myeloma cells by all-trans retinoic acid improves the efficacy of daratumumab. Leukemia. 2015; 29(10):2039-2049.
Nijhof I S, Groen R W, Noort W A, et al. Preclinical Evidence for the Therapeutic Potential of CD38-Targeted Immuno-Chemotherapy in Multiple Myeloma Patients Refractory to Lenalidomide and Bortezomib. Clin Cancer Res. 2015; 21(12):2802-2810.
Nijhof I S, Lammerts van Bueren J J, van Kessel B, et al. Daratumumab-mediated lysis of primary multiple myeloma cells is enhanced in combination with the human anti-KIR antibody IPH2102 and lenalidomide. Haematologica. 2015; 100(2):263-268.
Nooka A K, Joseph N S, Kaufman J L, et al. Clinical efficacy of daratumumab, pomalidomide, and dexamethasone in patients with relapsed or refractory myeloma: Utility of re-treatment with daratumumab among refractory patients. Cancer. 2019; 125(17):2991-3000.
O'Brien K L, Finlay D K. Immunometabolism and natural killer cell responses. Nat Rev Immunol. 2019; 19(5):282-290.
Orlowski R Z, Moreau P, Niesvizky R, et al. Carfilzomib-Dexamethasone Versus Bortezomib-Dexamethasone in Relapsed or Refractory Multiple Myeloma: Updated Overall Survival, Safety, and Subgroups. Clin Lymphoma Myeloma Leuk. 2019; 19(8):522-530 e521.
Pomeroy E J, Hunzeker J T, Kluesner M G, et al. A Genetically Engineered Primary Human Natural Killer Cell Platform for Cancer Immunotherapy. Mol Ther. 2020; 28(1):52-63.
Raab M S, Podar K, Breitkreutz I, Richardson P G, Anderson K C. Multiple myeloma. Lancet. 2009; 374(9686):324-339.
Rezvani K, Rouce R, Liu E, Shpall E. Engineering Natural Killer Cells for Cancer Immunotherapy. Mol Ther. 2017; 25(8):1769-1781.
Rezvani, K., Rouce, R., Liu, E. & Shpall, E. Engineering Natural Killer Cells for Cancer Immunotherapy. Mol Ther. 25 (8), 1769-1781, doi:10.1016/j.ymthe.2017.06.012, (2017).
Rosario M, Liu B, Kong L, et al. The IL-15-Based ALT-803 Complex Enhances FcgammaRIIIa-Triggered NK Cell Responses and In Vivo Clearance of B Cell Lymphomas. Clin Cancer Res. 2016; 22(3):596-608.
Saltarella I, Desantis V, Melaccio A, et al. Mechanisms of Resistance to Anti-CD38 Daratumumab in Multiple Myeloma. Cells. 2020; 9(1).
Sanchez-Martinez D, Krzywinska E, Rathore M G, et al. All-trans retinoic acid (ATRA) induces miR-23a expression, decreases CTSC expression and granzyme B activity leading to impaired NK cell cytotoxicity. Int J Biochem Cell Biol. 2014; 49:42-52.
Schultz, L. M., Majzner, R., Davis, K. L. & Mackall, C. New developments in immunotherapy for pediatric solid tumors. Curr Opin Pediatr. 30 (1), 30-39, doi:10.1097/mop.0000000000000564, (2018).
Somanchi, S. S., Senyukov, V. V., Denman, C. J. & Lee, D. A. Expansion, purification, and functional assessment of human peripheral blood NK cells. J Vis Exp. (48), doi:10.3791/2540, (2011).
Stevison F, Jing J, Tripathy S, Isoherranen N. Role of Retinoic Acid-Metabolizing Cytochrome P450s, CYP26, in Inflammation and Cancer. Adv Pharmacol. 2015; 74:373-412.
Sutlu, T. et al. Inhibition of intracellular antiviral defense mechanisms augments lentiviral transduction of human natural killer cells: implications for gene therapy. Hum Gene Ther. 23 (10), 1090-1100, doi:10.1089/hum.2012.080, (2012).
Uruno A, Noguchi N, Matsuda K, et al. All-trans retinoic acid and a novel synthetic retinoid tamibarotene (Am80) differentially regulate CD38 expression in human leukemia HL-60 cells: possible involvement of protein kinase C-delta. J Leukoc Biol. 2011; 90(2):235-247.
Usmani S Z, Weiss B M, Plesner T, et al. Clinical efficacy of daratumumab monotherapy in patients with heavily pretreated relapsed or refractory multiple myeloma. Blood. 2016; 128(1):37-44.
Vakulskas C A, Dever D P, Rettig G R, et al. A high-fidelity Cas9 mutant delivered as a ribonucleoprotein complex enables efficient gene editing in human hematopoietic stem and progenitor cells. Nat Med. 2018; 24(8):1216-1224.
van de Donk N, Usmani S Z. CD38 Antibodies in Multiple Myeloma: Mechanisms of Action and Modes of Resistance. Front Immunol. 2018; 9:2134.
van de Donk N W, Janmaat M L, Mutis T, et al. Monoclonal antibodies targeting CD38 in hematological malignancies and beyond. Immunol Rev. 2016; 270(1):95-112.
van der Veer M S, de Weers M, van Kessel B, et al. Towards effective immunotherapy of myeloma: enhanced elimination of myeloma cells by combination of lenalidomide with the human CD38 monoclonal antibody daratumumab. Haematologica. 2011; 96(2):284-290.
Viel, S. et al. TGF-beta inhibits the activation and functions of NK cells by repressing the mTOR pathway. Sci Signal. 9 (415), ra19, doi:10.1126/scisignal.aad1884, (2016).
Vouillot, L., Thelie, A. & Pollet, N. Comparison of T7E1 and surveyor mismatch cleavage assays to detect mutations triggered by engineered nucleases. G3 (Bethesda). 5 (3), 407-415, doi:10.1534/g3.114.015834, (2015).
Wang Y, Zhang Y, Hughes T, et al. Fratricide of NK Cells in Daratumumab Therapy for Multiple Myeloma Overcome by Ex Vivo-Expanded Autologous NK Cells. Clin Cancer Res. 2018; 24(16):4006-4017.
Wang, M. et al. Efficient delivery of genome-editing proteins using bioreducible lipid nanoparticles. Proc Natl Acad Sci USA. 113 (11), 2868-2873, doi:10.1073/pnas.1520244113, (2016).
Xu X S, Dimopoulos M A, Sonneveld P, et al. Pharmacokinetics and Exposure-Response Analyses of Daratumumab in Combination Therapy Regimens for Patients with Multiple Myeloma. Adv Ther. 2018; 35(11):1859-1872.
Zent C S, Secreto C R, LaPlant B R, et al. Direct and complement dependent cytotoxicity in CLL cells from patients with high-risk early-intermediate stage chronic lymphocytic leukemia (CLL) treated with alemtuzumab and rituximab. Leuk Res. 2008; 32(12):1849-1856.
Zuris, J. A. et al. Cationic lipid-mediated delivery of proteins enables efficient protein-based genome editing in vitro and in vivo. Nat Biotechnol. 33 (1), 73-80, doi:10.1038/nbt.3081, (2015).

1. A method of treating a cancer in a subject comprising administering to the subject a natural killer (NK) cell that has been modified to comprise a knockout of the clustering of differentiation 38 (CD38) gene.

2. The method of treating a cancer of claim 1, wherein the cancer comprises multiple myeloma, acute myeloid leukemia (AML), T-cell acute lymphoblastic leukemia (T-ALL), or Blastic plasmacytoid dendritic cell neoplasm (BPDCN).

3. The method of treating a cancer of claim 1, further comprising administering to the subject an anti-cancer agent that targets CD38, wherein the anti-cancer agent comprises a small molecule, an antibody, a peptide, a protein, or an siRNA.

4. The method of treating a cancer of claim 3, wherein the anti-cancer agent comprises Daratumumab, isatuximab, TAK-079, or MOR202.

5. The method of treating a cancer of claim 3, further comprising administering to the subject an angiogenesis inhibitor and a steroid.

6. The method of treating a cancer of claim 5, wherein the angiogenesis inhibitor comprises Pomalidomide, Lenalidomide, or Apremilast.

7. The method of treating a cancer of claim 5, wherein the steroid comprises a glucocorticoid.

8. The method of treating a cancer of claim 7, wherein the glucocorticoid comprises dexamethasone, betamethasone, prednisolone, methylprednisolone, triamcinolone, or fludrocortisone acetate.

9. A method of adoptively transferring an engineered natural killer (NK) cell to a subject in need thereof, said method comprising
a) obtaining a target NK cell to be modified;
b) obtaining gRNA specific for the target DNA sequence;
c) introducing via electroporation into the target NK cell, a RNP complex comprising a class 2 CRISPR/Cas endonuclease (Cas9) complexed with a corresponding CRISPR/Cas guide RNA that hybridizes to a target sequence within the genomic DNA of the target NK cell thereby creating an engineered NK cell; and
d) transferring the engineered NK cell into the subject.

10. The method of claim 9, wherein the subject has a cancer.

11. The method of claim 9, wherein the NK cell is a primary NK cell that has been modified ex vivo and after modification transferred to the subject.

12. The method of claim 9, wherein the NK cell is an autologous NK cell or obtained from an allogeneic donor source.

13. (canceled)

14. The method of claim 9, wherein the NK cell is expanded with irradiated mbIL-21 expressing feeder cells prior to administration to the subject.

15. The method of claim 9, wherein the NK cell is expanded in the subject following transfer of the NK cells to the subject via the administration of IL-21 or irradiated mbIL-21 expressing feeder cells.

16. The method of claim 9, wherein the RNP complex targets the clustering of differentiation 38 (CD38) gene.

17. A genetically modified NK cell comprising a knockout of the gene encoding clustering of differentiation 38 (CD38) made by the method of claim 9.

18. A method of treating a cancer in a subject comprising administering to the subjected the genetically modified NK cell of claim 17.

19. The method of claim 18, further comprising administering to the subject an anti-cancer agent that targets CD38, wherein the anti-cancer agent comprises a small molecule, an antibody, a peptide, a protein, or an siRNA.

20. (canceled)

21. The method of treating a cancer of claim 19, wherein the anti-CD38 agent comprises Daratumumab, isatuximab, TAK-079, or MOR202.

22. The method of treating a cancer of claim 18, wherein the cancer comprises multiple myeloma, acute myeloid leukemia (AML), T-cell acute lymphoblastic leukemia (T-ALL), or Blastic plasmacytoid dendritic cell neoplasm (BPDCN).

23. A method of reducing NK cell fratricide in a subject receiving anti-CD38 immunotherapy comprising administering to the subject the genetically modified NK cell of claim 17.


SEQUENCE LISTING (12 sequences)

SEQ ID NO: 1 - DNA, 20 bp, Artificial Sequence
  Synthetic construct
ctgaactcgcagttggccat

SEQ ID NO: 2 - DNA, 20 bp, Artificial Sequence
  Synthetic construct
ggggccactagggacaggat

SEQ ID NO: 3 - DNA, 20 bp, Artificial Sequence
  Synthetic construct
aaagagacttggtacccagg

SEQ ID NO: 4 - DNA, 20 bp, Artificial Sequence
  Synthetic construct
aaagagacttggtacccagg

SEQ ID NO: 5 - DNA, 29 bp, Artificial Sequence
  Synthetic construct
ccttggagtcgcagctagcctctgtgagg

SEQ ID NO: 6 - DNA, 55 bp, Artificial Sequence
  Synthetic construct
ccccgcctggagccctatggccaactgcgagttcagcccggtgtccggggacaaa

SEQ ID NO: 7 - DNA, 11 bp, Artificial Sequence
  Synthetic construct
tttattagtag

SEQ ID NO: 8 - DNA, 10 bp, Artificial Sequence
  Synthetic construct
ccctagagtt

SEQ ID NO: 9 - DNA, 10 bp, Artificial Sequence
  Synthetic construct
aaactctagg

SEQ ID NO: 10 - DNA, 22 bp, Artificial Sequence
  Synthetic construct
gggcaccggcaggaaagacaac

SEQ ID NO: 11 - DNA, 20 bp, Artificial Sequence
  Synthetic construct
accaacgaactgtaagggct

SEQ ID NO: 12 - DNA, 10 bp, Artificial Sequence
  Synthetic construct
tgcagaagaa